(19) United States
(12) Patent Application Publication
(10) Pub. No.: US 2023/0340041 A1
(43) Pub. Date: Oct. 26, 2023

(54) SHANK3 GENE THERAPY APPROACHES

(71) Applicant: Massachusetts Institute of Technology, Cambridge, MA (US)

(72) Inventors: Guoping Feng, Cambridge, MA (US); Xian Gao, Cambridge, MA (US); Yuan Mei, Cambridge, MA (US)

(73) Assignee: Massachusetts Institute of Technology, Cambridge, MA (US)

(21) Appl. No.: 18/021,693

(22) PCT Filed: Aug. 17, 2021
(86) PCT No.: PCT/US2021/046382

§ 371 Date: Feb. 16, 2023

Related U.S. Application Data
(60) Provisional application No. 63/066,570, filed on Aug. 17, 2020.

(51) Int. Cl. C07K 14/47; A61K 48/00; A61K 9/00; C12N 15/86

(52) U.S. Cl. CPC C07K 14/4702; A61K 9/0019; A61K 48/005; C12N 15/86; C12N 2750/14143

(57) ABSTRACT

Aspects of the disclosure relate to non-naturally occurring polynucleotides encoding a Shank3 protein, AAV vectors comprising the polynucleotides, and gene therapy methods.

### CROSS REFERENCE TO RELATED APPLICATIONS

This application claims the benefit under 35 U.S.C. § 119(e) of U.S. Provisional Application No. 63/066,570, filed Aug. 17, 2020, entitled “SHANK3 GENE THERAPY APPROACHES,” the entire disclosure of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Aug. 16, 2021, is named B119570108WO00-SEQ-SXT, and is 151,434 bytes in size.

### FIELD OF THE INVENTION

The present disclosure relates to gene therapy approaches for delivering polynucleotides encoding a Shank3 protein to a subject who has, is suspected of having, or is at risk of having, a neurodevelopmental disorder.

### BACKGROUND

Deletions and/or mutations involving Shank3 account for about 0.5-1% of all autism spectrum disorder (ASD) patients and about 2% ASD patients with intellectual disability (ID). However, there is no effective treatment for ASD and/or ID. Several challenges have arisen to developing pharmacological treatments that could correct the multitude of pathologies associated with ASD and ID.

### SUMMARY

Aspects of the disclosure relate to the development of an effective gene therapy approach for subjects with Shank3 mutations.

Aspects of the disclosure relate to non-naturally occurring polynucleotides encoding a Shank3 protein, wherein the Shank3 protein comprises an SH3 domain, a PDZ domain, a Homer binding domain, a Cortactin binding domain, and a SAM domain. In some embodiments, the SH3 domain comprises at least 90% identity to residues 474-525 of SEQ ID NO: 6 or at least 90% identity to residues 473-524 of SEQ ID NO: 5. In some embodiments, the PDZ domain comprises at least 90% identity to residues 573-662 of SEQ ID NO: 6 or at least 90% identity to residues 572-661 of SEQ ID NO: 5. In some embodiments, the Homer binding domain comprises at least 90% identity to residues 1294-1323 of SEQ ID NO: 5 or 6. In some embodiments, the Cortactin binding domain comprises at least 90% identity to residues 1400-1426 of SEQ ID NO: 5 or 6 and/or. In some embodiments, the SAM domain comprises at least 90% identity to residues 1664-1729 of SEQ ID NO: 6 or at least 90% identity to residues 1663-1728 of SEQ ID NO:5. In some embodiments, the polynucleotide is less than 4.7 kb.

In some embodiments, the polynucleotide further comprises a proline-rich region. In some embodiments, the SH3 domain comprises residues 474-525 of SEQ ID NO: 6 or residues 473-524 of SEQ ID NO: 5, the PDZ domain comprises residues 573-662 of SEQ ID NO: 6 or residues 572-661 of SEQ ID NO: 5, the Homer binding domain comprises residues 1294-1323 of SEQ ID NO: 5 or 6; the Cortactin binding domain comprises residues 1400-1426 of SEQ ID NO: 5 or 6 and/or the SAM domain comprises residues 1664-1729 of SEQ ID NO: 6 or residues 1663-1728 of SEQ ID NO:5.

In some embodiments, the polynucleotide comprises at least 90% identity to SEQ ID NO: 1 or 2. In some embodiments, the polynucleotide comprises SEQ ID NO: 1 or 2.

In some embodiments, the Shank3 protein encoded by the polynucleotide further comprises an ankyrin repeat domain. In some embodiments, the ankyrin repeat domain comprises at least 90% identity to residues 148-345 of SEQ ID NO: 6 or at least 90% identity to residues 147-313 of SEQ ID NO: 5. In some embodiments, the polynucleotide comprises residues 148-345 of SEQ ID NO: 6 or residues 147-313 of SEQ ID NO: 5.

In some embodiments, the polynucleotide comprises at least 90% identity to SEQ ID NO: 3 or 4. In some embodiments, the polynucleotide comprises SEQ ID NO: 3 or 4.

In some embodiments, the polynucleotide is less than about 4.6 kb, 4.5 kb, 4.4 kb, 4.3 kb, 4.2 kb, 4.1 kb, 4.0 kb, 3.9 kb, 3.8 kb, 3.7 kb, 3.6 kb, 3.5 kb, 3.4 kb, 3.3 kb, 3.2 kb, 3.1 kb, 3.0 kb, 2.9 kb, 2.8 kb, 2.7 kb, 2.6 kb, 2.5 kb, 2.4 kb, 2.3 kb, 2.2 kb, or 2.1 kb.

In some embodiments, the Shank3 protein is less than 65% identical to SEQ ID NO: 5 or 6 over the full length of SEQ ID NO: 5 or 6.

In some embodiments, the Shank3 protein comprises an amino acid sequence that is at least 90% identical to any one of SEQ ID NOs: 17-20. In some embodiments, the Shank3 protein comprises an amino acid sequence comprising any one of SEQ ID NOs: 17-20.

Further aspects of the disclosure relate to Shank3 proteins encoded by polypeptides described herein.

Further aspects of the disclosure relate to vectors comprising polynucleotides described herein. In some embodiments, the vector is a viral vector. In some embodiments, the vector is an AAV vector. In some embodiments, the vector comprises a promoter operably linked to the polynucleotide described herein. In some embodiments, the polynucleotide is flanked by AAV inverted terminal repeat (ITRs). In some embodiments, the AAV vector comprises a sequence that is at least 90% identical to SEQ ID NO: 7 or 21 and encodes a protein with Shank3 activity. In some embodiments, the AAV vector comprises the sequence of SEQ ID NO: 7 or 21 and encodes a protein with Shank3 activity. In some embodiments, the AAV vector comprises a sequence that is at least 90% identical to SEQ ID NO: 2 or 4 and encodes a protein with Shank3 activity. In some embodiments, the AAV vector comprises the sequence of SEQ ID NO: 2 or 4, which encodes a protein with Shank3 activity. In some embodiments, the AAV vector comprises a sequence that is at least 90% identical to SEQ ID NO: 1 or 3 and encodes a protein with Shank3 activity. In some embodiments, the AAV vector comprises the sequence of SEQ ID NO: 1 or 3, which encodes a protein with Shank3 activity.

Further aspects of the disclosure relate to AAV particles comprising an AAV vector and a capsid protein, wherein the capsid is of a serotype selected from AAV1, 2, 5, 6, 8, 9, rh10, and PHP.eB. In some embodiments, the serotype is AAV9. In some embodiments, the serotype is AAV10. In some embodiments, the serotype is PHP.eB.

In some embodiments, the AAV vector further comprises a promoter. In some embodiments, the promoter is a human promoter. In some embodiments, the promoter is hSyn1.

Further aspects of the disclosure relate to methods comprising administering AAV vectors or particles described herein to a subject in need thereof. In some embodiments, the subject is a human subject. In some embodiments, the human subject is an adult. In some embodiments, the human subject is not an adult. In some embodiments, the human subject is not older than 25 years old. In some embodiments, the human subject is 10 years old or younger. In some embodiments, the subject has, is suspected of having, or is at risk of having, a neurodevelopmental disorder. In some embodiments, the subject has, is suspected of having, or is at risk of having, an autism spectrum disorder (ASD). In some embodiments, the subject exhibits one or more symptoms of an ASD. In some embodiments, the subject has, is suspected of having, or is at risk of having, Phelan-McDermid syndrome. In some embodiments, the subject exhibits one or more of: developmental delay, intellectual disability (ID), sleep disturbance, hypotonia, lack of speech, or language delay.

Further aspects of the disclosure relate to methods of treating a subject having a neurodevelopmental disorder. In some embodiments, the disclosure relates to methods of treating a subject having an autism spectrum disorder (ASD). In some embodiments, the disclosure relates to methods of treating a subject having Phelan-McDermid syndrome. In some embodiments, the methods of treatment comprise administering to the subject an effective amount of a composition comprising an AAV vector that comprises a polynucleotide encoding a Shank3 protein. In some embodiments, the composition is in a pharmaceutically acceptable carrier.

In some embodiments, the AAV vector is delivered to the brain of the subject. In some embodiments, the AAV vector is delivered to the cortex, striatum and/or thalamus of the subject.

In some embodiments, the subject has, is suspected of having, or is at risk of having, reduced expression of the Shank3 gene relative to a control subject. In some embodiments, the control subject is a subject that does not have, is not suspected of having, or is not at risk of having, a neurodevelopmental disorder, an autism spectrum disorder (ASD), and/or Phelan-McDermid syndrome. In some embodiments, reduced expression of the Shank3 gene is caused by disruption of at least one copy of the Shank3 gene. In some embodiments, disruption of the Shank3 gene comprises a deletion in at least one copy of the Shank3 gene. In some embodiments, disruption of the Shank3 gene comprises one or more mutations within at least one copy of the Shank3 gene.

Further aspects of the disclosure relate to MiniShank3 proteins comprising a sequence that is at least 80%, at least 85%, at least 90%, or at least 95% identical to any one of SEQ ID NOs: 17-20. In some embodiments, the MiniShank3 protein comprises the sequence of any one of SEQ ID NOs: 17-20.

### BRIEF DESCRIPTION OF THE DRAWINGS

FIGS. 1A-1B show skin lesions and increased grooming in Shank3B^{-/-} mice.

FIGS. 2A-2C show that Shank3B mutant mice showed social interaction deficits. “Stranger 1” in FIG. 2A represents the partners that were tested for the social contact behaviors with the test animals. “Stranger 2” in FIG. 2A represents novice social partners that were introduced into the previously empty wired cage. S1: Stranger 1; S2: Stranger 2; E: Empty cage.

FIGS. 3A-3C show that Shank3B^{-/-} mice showed altered molecular composition in the striatal PSD.

FIGS. 4A-4E show cortico-striatal synaptic defects in Shank3B mutant mice. PPR: paired-pulse ratio.

FIGS. 5A-5C show the design of miniShank3-v1. FIG. 5A shows a protein domain diagram of the full length Shank3. FIG. 5B shows a schematic diagram of miniShank3-v1 provided in this disclosure. FIG. 5C shows a schematic diagram of GFP-tagged miniShank3-v1 with human synapsin-1 promoter (hSyn-1). ANK, ankyrin repeats; SH3, src homology 3 domain; PDZ, PDZ domain; Pro, proline rich region; HBD, Homer binding domain; CBD, Cortactin binding domain; SAM, sterile alpha motif.

FIGS. 6A-6C show the design of miniShank3-v2. FIG. 6A shows a protein domain diagram of the full length Shank3. FIG. 6B shows a schematic diagram of miniShank3-v2 provided in this disclosure. FIG. 6C shows a schematic diagram of GFP-tagged miniShank3-v2 with human synapsin-1 promoter (hSyn-1). ANK, ankyrin repeats; SH3, src homology 3 domain; PDZ, PDZ domain; Pro, proline rich region; HBD, Homer binding domain; CBD, Cortactin binding domain; SAM, sterile alpha motif.

FIGS. 7A-7H show that GFP-miniShank3-v1 was localized to synapses. hSyn1-GFP-miniShank3-v1 was transfected into striatal medium spiny neurons (MSN) in cortico-striatal coculture. FIG. 7A shows that GFP-miniShank3-v1 was expressed in MSN; FIGS. 7B-7C show the same culture stained with PSD95 to mark synapses (FIG. 7B) and with MAP2 to show dendrites (FIG. 7C). FIG. 7D shows merged images of FIGS. 7A-7C. FIGS. 7E-7H show high magnification images from FIGS. 7A-7D to show precise localization of GFP-miniShank3-v1 (FIG. 7E) with PSD95 (FIG. 7F) on dendrites (FIG. 7G) and dendritic spines in a merged image (FIG. 7H).

FIG. 8 shows that there was efficient expression of GFP-miniShank3-v1 after postnatal day 0 facial vein injection. P0 mice were injected with 6.42E+11 viral genomes (vg) per mouse. Mouse brains were collected 2 months after AAV injection and brains were sectioned to observe GFP expression.

FIGS. 9A-9F show that single intravenous injection of AAV-miniShank3-v1 at P0 rescued PSD protein deficits in Shank3-difficient mice. FIG. 9A shows a timeline of the AAV-mediated miniShank3-v1 gene therapy experiment and experimental groups. FIG. 9B provides a Western blot showing miniShank3 expression in striatal synaptosome plasma membrane (SPM) fractions prepared from wildtype mice injected with AAV-GFP (WT), InsG3680+/+ mice injected with AAV-GFP (Mutant), InsG3680+/+ mice injected with AAV-GFP-miniShank3-v1 (miniShank3), lysate from HEK293 cells expressing cDNA plasmid encoding GFP-miniShank3-v1 (HEK293-a), and HEK293 cells expressing cDNA plasmid encoding GFP-p2A miniShank3-v1 (HEK 293-b), which was detected using an anti-Shank3 antibody. FIGS. 9C and 9E show representative blots for proteins detected by specific antibodies in the striatal (FIG. 9C) and cortical (FIG. 9E) SPM fraction from AAV-injected mice: WT mice injected with AAV-GFP (WT), InsG3680+/+ mice injected with AAV-GFP (Mutant), and InsG3680+/+ mice injected with AAV-GFP-miniShank3-v1 (miniShank3). FIGS. 9D and 9F show quantification of relative levels of proteins as normalized to tubulin protein expression from striatal (FIG. 9D) and cortical (FIG. 9F) SPM. (n=4 samples per protein per genotype, each n being pooled tissue from two mice). Note that PSD protein levels in mutants were restored to WT levels in the Minishank3 group. *p<0.05, **p<0.01, ***p<0.001, one-way ANOVA with Bonferroni post-hoc test (FIGS. 9D and 9F).

FIGS. 10A-10B show that a single intravenous injection of AAV-miniShank3-v1 at P0 rescued the striatal synaptic defect in Shank3-difficient mice. FIG. 10A shows that striatal pop spikes amplitude reduced in mutant mice was rescued in animals injected with miniShank3-v1. FIG. 10B shows representative cortical-striatal pop spikes traces from mice with indicated treatment.

FIGS. 11A-11E show that systemic delivery of miniShank3-v1 at P0 rescued the behavioral deficits in Shank3-deficient mice. FIG. 11A shows that in the social interaction test, mutant mice displayed no preference with a stranger mice (S) over a novel object (O) compared to controls. This behavior was rescued by miniShank3 treatment. FIG. 11B shows that miniShank3 treatment rescued lower motor activity (decreased travel distance in open field test) in mutant mice to wildtype levels. FIG. 11C shows that reduced explorative behavior (rearing time) in Shank3 mutant mice was restored to WT level miniShank3 treatment group. FIG. 11D shows that anxiety-like behavior (reduced open arm time in elevated zero maze test) in Shank3 mutant mice was also rescued in miniShank3 treatment group. FIG. 11E shows that the trend of improvement in motor skills (rotarod test) was seen in the miniShank3 treatment group compared to the Shank3 mutant group. *p<0.05, **p<0.01, ***p<0.001, ****p<0.0001, one-way ANOVA with Bonferroni post-hoc test (FIGS. 11A-11D), two-way ANOVA with Bonferroni post-hoc test (FIG. 11E). Data are presented as mean ± SEM (a: n=16 WT+GFP, n=12 Mutant+GFP and n=14 Mutant + miniShank3; b-e: n= 26 WT + GFP, n=29 Mutant + GFP and n=19 Mutant + miniShank3).

FIGS. 12A-12F show that miniShank3 treatment at postnatal day 28 (P28) selectively rescued social impairment and motor deficits in Shank3 mutant mice. FIG. 12A shows time spent on close social interaction with an object (O) versus stranger mouse (S) in the phase II social preference assay. FIG. 12B shows total distance traveled in the open-field test. FIG. 12C shows evaluation of motor learning assessed via latency to fall in the rotarod test. FIG. 12D shows evaluation of motor coordination assessed via latency to fall in the rotarod test. FIG. 12E shows quantification of the time spent in the open arms of elevated zero maze to assess anxiety-like behavior. FIG. 12F shows assessment of grooming time in 2-hour videotaping. *p<0.05, **p<0.01, ***p<0.001, ****p<0.0001, one-way ANOVA with Bonferroni post-hoc test (FIGS. 12A, 12B, 12E, and 12F), two-way ANOVA with Bonferroni post-hoc test (FIGS. 12C and 12D). Data are presented as mean ± SEM (FIG. 12A: n=21 WT, n=23 Mut, and n=18 MiniShank3; FIG. 12B: n= 14 WT, n=20 Mut, and n=13 MiniShank3; FIG. 12C: n=17 WT, n=14 Mut and n=10 MiniShank3; FIG. 12D: n=17 WT, n=14 Mut and n=10 MiniShan3k; FIG. 12E: n=14 WT, n=20 Mut and n=14 MiniShank3; FIG. 12F: n=15 WT, n=18 Mut and n=17 MiniShank3). WT indicates wild type; mut indicates Shanks3 mutant; MiniShank3 indicates Shank 3 mutant + miniShank3 treatment.

FIGS. 13A-13I show that miniShank3 treatment at postnatal day (P7) fully rescued all behavioral deficits as well as disrupted sleep in Shank3 mutant mice. FIG. 13A shows time spent on close social interaction with an object (O) versus stranger mouse (S) in the phase II social preference assay. FIG. 13B shows assessment of grooming time in 2-hour videotaping. FIG. 13C shows quantification of the time spent in the open arms of elevated zero maze to assess anxiety-like behavior. FIGS. 13D and 13E show total distance traveled in the open-field test. FIG. 13F shows evaluation of motor learning and coordination assessed via latency to fall in the rotarod test. FIG. 13G shows quantification of NREM sleep duration to assess sleep behavior in Shank3 mutant mice. FIG. 13H shows quantification of NREM sleep bout length to assess sleep behavior in Shank3 mutant mice. FIG. 131 shows quantification of delta power to assess sleep behavior in Shank3 mutant mice. *p<0.05, **p<0.01, ***p<0.001, ****p<0.0001, one-way ANOVA with Bonferroni post-hoc test (FIGS. 13A, 13B, 13C, 13D, 13G, 13H, and 13I), two-way ANOVA with Bonferroni post-hoc test (FIGS. 13E and 13F). Data are presented as mean ± SEM (FIG. 13A: n=16 WT, n=9 Mut, and n=14 MiniShank3; FIG. 13B: n= 18 WT, n=12 Mut, and n=14 MiniShank3; FIG. 13C: n=10 WT, n=10 Mut and n=10 MiniShank3; FIG. 13D: n=18 WT, n=14 Mut and n=18 MiniShank3; FIG. 13E: n=18 WT, n=14 Mut and n=18 MiniShank3; FIG. 13F: n=17 WT, n=15 Mut and n=17 MiniShank3; FIGS. 13G-13I: n=9 WT, n=8 Mut and n=8 MiniShank3). WT indicates wild type; mut indicates Shanks3 mutant; MiniShank3 indicates Shank3 mutant + miniShank3 treatment.

FIGS. 14A-14B show that miniShank3 treatment at postnatal day (P7) did not cause seizure activity in Shank3 mutants. FIG. 14A shows representative EEG traces of wild-type animals, mutants injected with control virus, mutants injected with miniShank3, or Scn2a mutants. FIG. 14B shows quantification of spike-wave discharges (SWDs) observed in the four experimental groups shown in panel a (data represented as mean ± SEM). ***p<0.001, one-way ANOVA with Bonferroni post-hoc test.

FIG. 15 illustrates a schematic showing plasmid construction of a human miniShank3 gene with a hSynl promoter.

### DETAILED DESCRIPTION

Aspects of the disclosure relate to gene therapy approaches for treating neurodevelopmental disorders. The Examples demonstrate non-naturally occurring polynucleotides encoding Shank3 proteins that can be expressed in gene delivery vectors and administered to subjects. Gene therapy strategies disclosed herein use an AAV system to deliver a functional copy of the Shank3 gene into brain cells to restore cellular function.

SHANK3 encodes synaptic scaffolding proteins at the excitatory glutamatergic synapses, coordinates the recruitment of signaling molecules and orchestrates assembly of the macromolecular postsynaptic protein complex, which is crucial for proper synaptic development and function. Deletion of SHANK3 is a major cause of the core neurodevelopmental and neurobehavioral deficits in Phelan-McDermid syndrome. Human genetic studies also identified SHANK3 mutations as accounting for about 1% of autism spectrum disorder (ASD). Patients with Phelan-McDermid syndrome and other individuals with SHANK3 mutations often exhibit a variety of comorbid traits, which include developmental delay, sleep disturbances, hypotonia, lack of speech or severe language delay, and characteristic features of ASD. Currently, there is no effective treatment for ASD.

The association of ASD with Shank3 provided an immediate link between synaptic dysfunction and the pathophysiology of ASD. Animal models bridge the human genetics of ASD to brain pathology underlying clinical presentation, and ultimately help to discover and evaluate effective therapeutics. Previous studies in flies, fish, and rodents have revealed synaptic dysfunction and behavioral abnormalities due to loss of SHANK3. For example, disruption of Shank3 in mouse models have resulted in synaptic defects, impaired social interactions, motor difficulties, repetitive grooming and increased anxiety level. Since Shank3 deficiency causes severe sleep disturbances in rodents, monkeys and human patients, sleep efficiency provides a unique biomarker for ASD. Furthermore, the Shank3-deficient mouse model presents predictive validity as the synaptic defects and behavioral abnormalities are reversible when Shank3 is restored. Therefore, gene replacement is well suited as a therapeutic strategy for this monogenic disease.

Novel recombinant adeno-associated viruses (rAAVs) represent a promising gene delivery platform because of their wide range of tissue tropism, low immunogenicity, highly efficient and sustained gene transduction, and clinically proven track record in safety. However, it is known in the art that Shank3 is a large protein with a coding sequence of about 5.7 kb, exceeding the packaging capacity of AAV vectors. The inventors of the instant application found that certain regions of the Shank3 protein are not critical for the function of the protein, and therefore, designed heterologous Shank3 expression constructs that have a significantly smaller coding sequence (about 2.1 kb to about 3.1 kb) because they encode for a version of the Shank3 protein that has certain non-essential regions removed. The resulting miniaturized Shank3 proteins described herein can be delivered by vector such as AAVs. The inventors of the instant application surprisingly found that a miniaturized Shank3 protein can restore defective functions caused by deletion or mutation of the gene encoding the Shank3 protein in a mouse model, and accordingly, can potentially rescue abnormalities caused by diseases that are associated with Shank3 mutations or deletions. This is in contrast with methods known in the art, which focus on repairing or improving partial fragments of the Shank3 protein but which are not able to restore the functions of the Shank protein. Thus, the present disclosure relates to methods and compositions for treating neurodevelopmental disorders by restoring the activity of Shank3 using a miniaturized Shank3 protein (“MiniShank3”).

### Shank Proteins

The Shank family of proteins (e.g., Shank1, Shank 2, and Shank3) are master scaffolding proteins that tether and organize scaffolding proteins at the synapses of excitatory neurons. Members of this family share at least five main domain regions: N-terminal ankyrin repeats, SH3 domain, PDZ domain, proline-rich region, and a C-terminal SAM domain. Through these functional domains, Shank proteins interact with many postsynaptic density (PSD) proteins. Without wishing to be bound by any theory, Shank proteins can bind to SAPAP which in turn binds to PSD95 to form the PSD95/SAPAP/Shank postsynaptic complex. Together, these multidomain proteins are proposed to form a key scaffold, orchestrating the assembly of the macromolecular postsynaptic signaling complex at glutamatergic synapses. This complex has been shown to play important roles in targeting, anchoring, and dynamically regulating synaptic localization of neurotransmitter receptors and signaling molecules. In another example, the Shank family of proteins is connected to the mGluR pathway through its binding to Homer.

Due to its link to actin-binding proteins, Shank also plays a major role in spine development. It has been found that transfection of Shank3 was sufficient to induce functional dendritic spine synapses in cultured aspiny cerebellar granule cells, indicating a role in spine induction. Shank3 has three primary isoforms including Shank3α, the longest Shank3 isoform, Shank3_{β} and Shank3_{γ}. It has been reported that siRNA knockdown of Shank3 reduced the number and increased the length of dendritic spines in DIV18 cultured hippocampal neurons, implicating a role in spine maturation. This proposed function was supported by the finding that overexpression of Shank1 enlarged already present dendritic spines in cultured hippocampal neurons. Furthermore, Shank1 mutant mice have been reported to have smaller dendritic spines and weaker synaptic transmission.

In some embodiments, the present disclosure relates to Shank proteins that are capable of restoring synaptic activity in subjects with disrupted Shank protein activity. In some embodiments, the disrupted Shank protein activity is present in subjects who have neurodevelopmental disorders, an autism spectrum disorder (ASD), and/or Phelan-McDermid syndrome. In some embodiments, the Shank proteins associated with the present disclosure are Shank1 proteins. In some embodiments, the present disclosure relates to expression in a subject in need thereof a polynucleotide encoding Shank1 or a variant of Shank1. In some embodiments, the Shank proteins in the present disclosure are Shank2 proteins. In some embodiments, the present disclosure relates to expression in a subject in need thereof a polynucleotide encoding Shank2 or a variant of Shank2. In some embodiments, the Shank proteins in the present disclosure are Shank3 proteins. In some embodiments, the present disclosure relates to expression in a subject in need thereof a polynucleotide encoding Shank3 or a variant of Shank3. It should be appreciated that Shank proteins associated with the present disclosure can include any Shank protein, including variants or fragments thereof, that function as scaffolding proteins at the synapses of excitatory neurons.

Also disclosed herein are polynucleotides encoding Shank proteins (Shank1, Shank 2, and Shank3) for use in gene therapy.

The Shank3 full length mouse protein sequence corresponding to GenBank Accession No. BAE16756.1 is provided by SEQ ID NO: 5:

MDGPGASAVVVRVGIPDLQQTKCLRLDPTAPVWAAKQRVLCALNHSLQDA
LNYGLFQPPSRGRAGKFLDEERLLQDYPPNLDTPLPYLEFRYKRRVYAQN
LIDDKQFAKLHTKANLKKFMDYVQLHSTDKVARLLDKGLDPNFHDPDSGE
CPLSLAAQLDNATDLLKVLRNGGAHLDFRTRDGLTAVHCATRQRNAGALT
TLLDLGASPDYKDSRGLTPLYHSALGGGDALCCELLLHDHAQLGTTDENG
WQEIHQACRFGHVQHLEHLLFYGANMGAQNASGNTALHICALYNQESCAR
VLLFRGANKDVRNYNSQTAFQVAIIAGNFELAEVIKTHKDSDVVPFRETP
SYAKRRRLAGPSGLASPRPLQRSASDINLKGDQPAASPGPTLRSLPHQLL
LQRLQEEKDRDRDGELENDISGPSAGRGGHNKISPSGPGGSGPAPGPGPA
SPAPPAPPPRGPKRKLYSAVPGRKFIAVKAHSPQGEGEIPLHRGEAVKVL
SIGEGGFWEGTVKGRTGWFPADCVEEVQMRQYDTRHETREDRTKRLFRHY
TVGSYDSLTSHSDYVIDDKVAILQKRDHEGFGFVLRGAKAETPIEEFTPT
PAFPALQYLESVDVEGVAWRAGLRTGDFLIEVNGVNVVKVGHKQVVGLIR
QGGNRLVMKVVSVTRKPEEDGARRRAPPPPKRAPSTTLTLRSKSMTAELE
ELASIRRRKGEKLDEILAVAAEPTLRPDIADADSRAATVKQRPTSRRITP
AEISSLFERQGLPGPEKLPGSLRKGIPRTKSVGEDEKLASLLEGRFPRST
SMQDTVREGRGIPPPPQTAPPPPPAPYYFDSGPPPTFSPPPPPGRAYDTV
RSSFKPGLEARLGAGAAGLYDPSTPLGPLPYPERQKRARSMIILQDSAPE
VGDVPRPAPAATPPERPKRRPRPSGPDSPYANLGAFSASLFAPSKPQRRK
SPLVKQLQVEDAQERAALAVGSPGPVGGSFAREPSPTHRGPRPGSLDYSS
GEGLGLTFGGPSPGPVKERRLEERRRSTVFLSVGAIEGSPPSADLPSLQP
SRSIDERLLGTGATTGRDLLLPSPVSALKPLVGGPSLGPSGSTFIHPLTG
KPLDPSSPLALALAARERALASQTPSRSPTPVHSPDADRPGPLFVDVQTR
DSERGPLASP AFSPRSP A WIPVP ARREAEKPPREERKSPEDKKSMI
LSVLDTSLQRPAGLIVVHATSNGQEPSRLGAEEERPGTPELAPAPMQAAA
VAEPMPSPRAQPPGSIPADPGPGQGSSEEEPELVFAVNLPPAQLSSSDEE
TREELARIGLVPPPEEFANGILLTTPPPGPGPLPTTVPSPASGKPSSELP
PAPESAADSGVEEADTRSSSDPHLETTSTISTVSSMSTLSSESGELTDTH
TSFADGHTFLLEKPPVPPKPKLKSPLGKGPVTFRDPLLKQSSDSELMAQQ
HHAASTGLASAAGPARPRYLFQRRSKLWGDPVESRGLPGPEDDKPTVISE
LSSRLQQLNKDTRSLGEEPVGGLGSLLDPAKKSPIAAARLFSSLGELSTI
SAQRSPGGPGGGASYSVRPSGRYPVARRAPSPVKPASLERVEGLGAGVGG
AGRPFGLTPPTILKSSSLSIPHEPKEVRFVVRSVSARSRSPSPSPLPSPS
PGSGPSAGPRRPFQQKPLQLWSKFDVGDWLESIHLGEHRDRFEDHEIEGA
HLPALTKEDFVELGVTRVGHRMNIERALRQLDGS

In some embodiments, the Shank3 full length mouse protein sequence corresponding to SEQ ID NO: 5 is encoded by a nucleic acid sequence corresponding to GenBank Accession No. NM_021423, provided by SEQ ID NO: 15:

atggacggccccggggccagcgccgtggtcgtgcgcgtcggcatcccgga
cctgcaacaaacgaagtgcctgcgtctggacccaaccgcgcccgtgtggg
ccgccaagcagcgtgtgctctgcgccctcaatcatagccttcaagacgcg
ctcaactacgggctattccagcctccctcccggggtcgcgccggcaagtt
cctggatgaagagcggctcttacaggactacccgcctaacctggacacgc
ccctgccctatctggagttccgatacaagcggagagtttatgcccagaac
ctcatagatgacaagcagtttgcaaagctacacacaaaggcaaacctgaa
gaagttcatggactatgtccagctacacagcacagataaggtggcccgcc
tgctggacaaggggctggaccccaatttccatgaccctgactcaggagag
tgccctctgagccttgcggcacagttggacaacgccactgacctcctgaa
ggttctccgcaacggcggtgctcatctggacttccggacccgagatgggc
tgacagccgtccactgtgctacccgccagcggaacgcaggggcattgacg
accctgctggacctgggggcttcgcctgactacaaggacagccgcggcct
gacgcccctgtaccatagtgccctagggggcggggatgccctctgttgcg
agctgcttctccatgatcatgcacagctggggaccactgatgagaatggt
tggcaagagatccatcaggcctgtcgctttggacacgtgcagcacctgga
gcaccttttgttctatggggccaacatgggtgctcagaatgcctcgggaa
acacagccctgcacatctgtgccctctacaaccaggagagttgcgcgcgc
gtcctgcttttccgtggtgccaacaaggacgtccgcaattacaacagcca
gacagccttccaggtggccattattgcagggaactttgagcttgccgagg
taatcaagacccacaaagactccgatgtcgtaccattcagggaaaccccc
agctatgcaaagcgacggcgtctggctggcccgagtggcctggcatcccc
acggcccttacagcgctcagccagtgatatcaacctgaaaggtgatcagc
ccgcagcttctccagggcccactctccgaagcctccctcatcaactcttg
ctccagaggcttcaggaggagaaagaccgtgacagggatggtgaactgga
gaatgacatcagcggcccctcagcaggcaggggtggccacaacaagatca
gccccagtgggcccggcggatccggccccgcgcccggccccggcccggcg
tctcccgcgccccccgcgccgccgccccggggcccgaagcggaaacttta
cagtgccgtccccggccgcaagttcatcgctgtgaaggcgcacagcccgc
agggcgagggcgagatcccgctgcaccgcggcgaggccgtgaaggtgctc
agcattggggagggcggtttctgggagggaaccgtgaagggccgaacagg
ctggttcccagctgactgtgtggaagaagtgcagatgcgacagtatgaca
cccggcatgaaaccagagaggaccggacgaagcgtctcttccgccactac
actgtgggttcctatgacagcctcacttcacacagcgattatgtcatcga
tgataaggtggctatcctgcagaaaagggaccatgaggggtttggctttg
ttctccggggagccaaagcagagacccccattgaggagtttacacccaca
cctgccttccctgcactccaataccttgagtctgtagatgtggaaggtgt
ggcctggagggctggacttcgaactggggacttcctcattgaggtgaacg
gagtgaatgtcgtgaaggttggacacaagcaagtggtgggtctcatccgt
cagggtggcaaccgcctggtcatgaaggttgtgtctgtgaccaggaaacc
cgaggaggatggtgctcggcgcagagccccaccacccccaaagagggctc
ccagcaccacgctgaccctgcggtccaagtccatgacggctgagctcgag
gaacttgcttccattcggagaagaaaaggggagaagttggatgagatcct
ggcagttgccgcggagccgacactgaggccggacattgcagatgctgact
cgagggcggccactgtcaagcagcggcccaccagccggaggatcacccct
gctgagatcagctcattgtttgagcgccagggcctcccaggcccagagaa
gctgccgggctctctgcggaaggggattccacggaccaaatctgtagggg
aggatgagaagctggcatccctactggaagggcgcttcccacgcagcacg
tcaatgcaggacacagtgcgtgaaggtcgaggcattccacccccgccgca
gaccgccccgccacccccacccgcgccctactacttcgactccgggccac
cccccaccttctcaccgccgccaccaccgggccgggcctatgacactgtg
cgctccagcttcaaaccaggcctggaggctcgtctgggtgcgggggccgc
cggcctgtatgatccgagcacgcctctgggcccgctgccctaccctgagc
gtcagaagcgtgcgcgctccatgatcatactgcaggactctgcgccagaa
gtgggtgatgtcccccggcctgcgcctgccgccacaccgcctgagcgccc
caagcgccggcctcggccgtcaggccctgatagtccctatgccaacctgg
gcgccttcagtgccagcctcttcgctccgtcgaaaccacagcgccgcaag
agcccgctggtgaagcagcttcaggtggaggacgctcaggagcgcgcggc
cctggccgtgggtagcccgggaccagtgggcggaagctttgcacgagaac
cctctccgacgcaccgcgggccccggccaggcagccttgactacagctct
ggagaaggcctaggccttaccttcggcggccctagccctggcccagtcaa
ggagcggcgcctggaggagcgacgccgttccactgtgttcctctctgtgg
gtgccatcgagggcagccctcccagcgcggatctgccatccctacaaccc
tcccgctccattgatgagcgcctcctggggacaggcgccaccactggccg
cgatttgctactcccctcccctgtctctgctctgaagccattggtcggtg
gtcccagccttgggccctcaggctccaccttcatccaccctctcactggc
aaacccttggatcctagctcacccttagctcttgctctggctgcccggga
gcgggctctggcctcgcaaacaccttcccggtcccccacacctgtgcaca
gccccgatgctgaccgccctggacccctctttgtggatgtgcaaacccga
gactctgagagaggaccgttggcttccccagccttctcccctcggagtcc
agcgtggattccagtgcctgctcggagagaggcagagaagccccctcggg
aagagcggaagtcaccagaggacaagaagtccatgatcctcagcgtcttg
gacacgtccttgcaacggccagctggcctcattgttgtgcatgccaccag
caatgggcaggagcccagcaggctgggggctgaagaggagcgccccggta
ctccggagctggccccagcccccatgcaggcagcagctgtggcagagccc
atgccaagcccccgggcccagccccctggcagcatcccagcagatcccgg
gccaggtcaaggcagctcagaggaggagccagagctggtattcgctgtga
acctgccacctgctcagctgtcctccagcgatgaggagaccagagaggag
ctggcccgcatagggctagtgccaccccctgaagagtttgccaatgggat
cctgctgaccaccccgcccccagggccgggccccttgcccaccacggtac
ccagcccggcctcagggaagcccagcagcgagctgccccctgcccctgag
tctgcagctgactctggagtagaggaggctgacactcgaagctccagtga
cccccacctggagaccacaagcaccatttccacagtgtccagcatgtcca
ccctgagctcggagagtggggaactcacggacacccacacctcctttgcc
gatggacacacttttctactcgagaagccaccagtgcctcccaagcccaa
gctcaagtccccgctggggaaggggccggtgaccttcagggacccgctgc
tgaagcaatcctcggacagtgagctcatggcccagcagcaccatgctgcc
tctactgggttggcttctgctgctgggcccgcccgccctcgctacctctt
ccagagaaggtccaagctgtggggggaccccgtggagagtcgggggctcc
ctgggcctgaagatgacaaaccaactgtgatcagtgagctcagctcccgt
ctgcagcagctgaataaagacacacgctccttgggggaggaaccagttgg
tggcctgggcagcctgtggaccctgctaagaagtcacccattgcagcagc
tcggctcttcagcagcctcggtgagctgagcaccatctcagcgcagcgca
gcccggggggcccgggcggaggggcctcctactcggtgcggcccagcggc
cggtaccccgtggcgagacgagccccgagcccagtgaaacccgcatcgct
ggagcgggtggaggggctgggggcgggcgtgggaggcgcggggcggccct
tcggcctcacgcctcccaccatcctcaagtcgtccagcctctccatcccg
cacgaacccaaggaagtgcgcttcgtggtgcgaagtgtgagtgcgcgcag
ccgctccccctcaccatctccgctgccctcgccttctcccggctctggcc
ccagtgccggcccgcgtcggccatttcaacagaagcccctgcagctctgg
agcaagttcgatgtgggcgactggctggagagcatccacttaggcgagca
ccgagaccgcttcgaggaccatgagatcgaaggcgcacacctgcctgcgc
tcaccaaggaagacttcgtggagctgggcgtcacacgcgttggccaccgc
atgaacatcgagcgtgcgctcaggcagctggatggcagctga

The Shank3 full length human protein sequence corresponding to GenBank Accession No. Q9BYB0.3 is provided by SEQ ID NO: 6:

MDGPGASAVVVRVGIPDLQQTKCLRLDPAAPVWAAKQRVLCALNHSLQDA
LNYGLFQPPSRGRAGKFLDEERLLQEYPPNLDTPLPYLEFRYKRRVYAQN
LIDDKQFAKLHTKANLKKFMDYVQLHSTDKVARLLDKGLDPNFHDPDSGE
CPLSLAAQLDNATDLLKVLKNGGAHLDFRTRDGLTAVHCATRQRNAAALT
TLLDLGASPDYKDSRGLTPLYHSALGGGDALCCELLLHDHAQLGITDENG
WQEIHQACRFGHVQHLEHLLFYGADMGAQNASGNTALHICALYNQESCAR
VLLFRGANRDVRNYNSQTAFQVAIIAGNFELAEVIKTHKDSDVVPFRETP
SYAKRRRLAGPSGLASPRPLQRSASDINLKGEAQPAASPGPSLRSLPHQL
LLQRLQEEKDRDRDADQESNISGPLAGRAGQSKISPSGPGGPGPAPGPGP
APPAPPAPPPRGPKRKLYSAVPGRKFIAVKAHSPQGEGEIPLHRGEAVKV
LSIGEGGFWEGTVKGRTGWFPADCVEEVQMRQHDTRPETREDRTKRLFRH
YTVGSYDSLTSHSDYVIDDKVAVLQKRDHEGFGFVLRGAKAETPIEEFTP
TPAFPALQYLESVDVEGVAWRAGLRTGDFLIEVNGVNVVKVGHKQVVALI
RQGGNRLVMKVVSVTRKPEEDGARRRAPPPPKRAPSTTLTLRSKSMTAEL
EELASIRRRKGEKLDEMLAAAAEPTLRPDIADADSRAATVKQRPTSRRIT
PAEISSLFERQGLPGPEKLPGSLRKGIPRTKSVGEDEKLASLLEGRFPRS
TSMQDPVREGRGIPPPPQTAPPPPPAPYYFDSGPPPAFSPPPPPGRAYDT
VRSSFKPGLEARLGAGAAGLYEPGAALGPLPYPERQKRARSMIILQDSAP
ESGDAPRPPPAATPPERPKRRPRPPGPDSPYANLGAFSASLFAPSKPQRR
KSPLVKQLQVEDAQERAALAVGSPGPGGGSFAREPSPTHRGPRPGGLDYG
AGDGPGLAFGGPGPAKDRRLEERRRSTVFLSVGAIEGSAPGADLPSLQPS
RSIDERLLGTGPTAGRDLLLPSPVSALKPLVSGPSLGPSGSTFIHPLTGK
PLDPSSPLALALAARERALASQAPSRSPTPVHSPDADRPGPLFVDVQARD
PERGSLASPAFSPRSPAWIPVPARREAEKVPREERKSPEDKKSMILSVLD
TSLQRPAGLIVVHATSNGQEPSRLGGAEEERPGTPELAPAPMQSAAVAEP
LPSPRAQPPGGTPADAGPGQGSSEEEPELVFAVNLPPAQLSSSDEETREE
LARIGLVPPPEEFANGVLLATPLAGPGPSPTTVPSPASGKPSSEPPPAPE
SAADSGVEEADTRSSSDPHLETTSTISTVSSMSTLSSESGELTDTHTSFA
DGHTFLLEKPPVPPKPKLKSPLGKGPVTFRDPLLKQSSDSELMAQQHHAA
SAGLASAAGPARPRYLFQRRSKLWGDPVESRGLPGPEDDKPTVISELSSR
LQQLNKDTRSLGEEPVGGLGSLLDPAKKSPIAAARLFSSLGELSSISAQR
SPGGPGGGASYSVRPSGRYPVARRAPSPVKPASLERVEGLGAGAGGAGRP
FGLTPPTILKSSSLSIPHEPKEVRFVVRSVSARSRSPSPSPLPSPASGPG
PGAPGPRRPFQQKPLQLWSKFDVGDWLESIHLGEHRDRFEDHEIEGAHLP
ALTKDDFVELGVTRVGHRMNIERALRQLDGS

In some embodiments, the Shank3 full length human protein sequence corresponding to SEQ ID NO: 6 is encoded by a nucleic acid sequence corresponding to GenBank Accession No. NM_001372044, provided by SEQ ID NO: 16:

atgcagctgagccgcgccgccgccgccgccgccgccgcccctgcggagcc
cccggagccgctgtcccccgcgccggccccggccccggccccccccggcc
ccctcccgcgcagcgcggccgacggggctccggcgggggggaaggggggg
ccggggcgccgcgcggagtccccgggcgctccgttccccggcgcgagcgg
ccccggcccgggccccggcgcggggatggacggccccggggccagcgccg
tggtcgtgcgcgtcggcatcccggacctgcagcagacgaagtgcctgcgc
ctggacccggccgcgcccgtgtgggccgccaagcagcgcgtgctctgcgc
cctcaaccacagcctccaggacgcgctcaactatgggcttttccagccgc
cctcccggggccgcgccggcaagttcctggatgaggagcggctcctgcag
gagtacccgcccaacctggacacgcccctgccctacctggagtttcgata
caagcggcgagtttatgcccagaacctcatcgatgataagcagtttgcaa
agcttcacacaaaggcgaacctgaagaagttcatggactacgtccagctg
catagcacggacaaggtggcacgcctgttggacaaggggctggaccccaa
cttccatgaccctgactcaggagagtgccccctgagcctcgcagcccagc
tggacaacgccacggacctgctaaaggtgctgaagaatggtggtgcccac
ctggacttccgcactcgcgatgggctcactgccgtgcactgtgccacacg
ccagcggaatgcggcagcactgacgaccctgctggacctgggggcttcac
ctgactacaaggacagccgcggcttgacacccctctaccacagcgccctg
gggggtggggatgccctctgctgtgagctgcttctccacgaccacgctca
gctggggatcaccgacgagaatggctggcaggagatccaccaggcctgcc
gctttgggcacgtgcagcatctggagcacctgctgttctatggggcagac
atgggggcccagaacgcctcggggaacacagccctgcacatctgtgccct
ctacaaccaggagagctgtgctcgtgtcctgctcttccgtggagctaaca
gggatgtccgcaactacaacagccagacagccttccaggtggccatcatc
gcagggaactttgagcttgcagaggttatcaagacccacaaagactcgga
tgttgtaccattcagggaaacccccagctatgcgaagcggcggcgactgg
ctggccccagtggcttggcatcccctcggcctctgcagcgctcagccagc
gatatcaacctgaagggggaggcacagccagcagcttctcctggaccctc
gctgagaagcctcccccaccagctgctgctccagcggctgcaagaggaga
aagatcgtgaccgggatgccgaccaggagagcaacatcagtggcccttta
gcaggcagggccggccaaagcaagatcagcccgagcgggcccggcggccc
cggccccgcgcccggccccggccccgcgccccctgcgccccccgcaccgc
cgccccggggcccgaagcggaaactttacagcgccgtccccggccgcaag
ttcatcgccgtgaaggcgcacagcccgcagggtgaaggcgagatcccgct
gcaccgcggcgaggccgtgaaggtgctcagcattggggagggcggtttct
gggagggaaccgtgaaaggccgcacgggctggttcccggccgactgcgtg
gaggaagtgcagatgaggcagcatgacacacggcctgaaacgcgggagga
ccggacgaagcggctctttcggcactacacagtgggctcctacgacagcc
tcacctcacacagcgattatgtcattgatgacaaagtggctgtcctgcag
aaacgggaccacgagggctttggttttgtgctccggggagccaaagcaga
gacccccatcgaggagttcacgcccacgccagccttcccggcgctgcagt
atctcgagtcggtggacgtggagggtgtggcctggagggccgggctgcgc
acgggagacttcctcatcgaggtgaacggggtgaacgtggtgaaggtcgg
acacaagcaggtggtggctctgattcgccagggtggcaaccgcctcgtca
tgaaggttgtgtctgtgacaaggaagccagaagaggacggggctcggcgc
agagccccaccgccccccaagagggcccccgcaccacactgaccctgcgc
tccaagtccatgacagctgagctcgaggaacttgcctccattcggagaag
aaaaggggagaagctggacgagatgctggcagccgccgcagagccaacgc
tgcggccagacatcgcagacgcagactccagagccgccaccgtcaaacag
aggcccaccagtcggaggatcacacccgccgagattagctcattgtttga
acgccagggcctcccaggcccagagaagctgccgggctccttgcggaagg
ggattccacggaccaagtctgtaggggaggacgagaagctggcgtccctg
ctggaagggcgcttcccgcggagcacctcgatgcaagacccggtgcgcga
gggtcgcggcatcccgcccccgccgcagaccgcgccgcctcccccgcccg
cgccctactacttcgactcggggccgcccccggccttctcgccgccgccc
ccgccgggccgcgcctacgacacggtgcgctccagcttcaagcccggcct
ggaggcgcgcctgggcgcgggcgctgccggcctgtacgagccgggcgcgg
ccctcggcccgctgccgtatcccgagcggcagaagcgcgcgcgctccatg
atcatcctgcaggactcggcgcccgagtcgggcgacgcccctcgaccccc
gcccgcggccaccccgcccgagcgacccaagcgccggccgcggccgcccg
gccccgacagcccctacgccaacctgggcgccttcagcgccagcctcttc
gctccgtccaagccgcagcgccgcaagagccccctggtgaagcagctgca
ggtggaggacgcgcaggagcgcgcggccctggccgtgggcagccccggtc
ccggcggcggcagcttcgcccgcgagccctccccgacccaccgcggtccg
cgcccgggtggcctcgactacggcgcgggcgatggcccggggctcgcgtt
cggcggcccgggcccggccaaggaccggcggctggaggagcggcgccgct
ccactgtgttcctgtccgtgggggccatcgagggcagcgcccccggcgcg
gatctgccatccctacagccctcccgctccatcgacgagcgcctcctggg
gaccggccccaccgccggccgcgacctgctgctgccctccccggtgtctg
ccctgaagccgttggtcagcggcccgagcctggggccctcgggttccacc
ttcatccacccactcaccggcaaacccctggaccccagctcacccctggc
ccttgccctggctgcccgagagcgagctctggcctcccaggcgccctccc
ggtcccccacacccgtgcacagtcccgacgccgaccgccccggacccctg
tttgtggatgtacaggcccgggacccagagcgagggtccctggcttcccc
ggctttctccccacggagcccagcctggattcctgtgcctgctcgcaggg
aggcagagaaggtcccccgggaggagcggaagtcacccgaggacaagaag
tccatgatcctcagcgtcctggacacatccctgcagcggccagctggcct
catcgttgtgcacgccaccagcaacgggcaggagcccagcaggctggggg
gggccgaagaggagcgcccgggcaccccggagttggccccggcccccatg
cagtcagcggctgtggcagagcccctgcccagcccccgggcccagccccc
tggtggcaccccggcagacgccgggccaggccagggcagctcagaggaag
agccagagctggtgtttgctgtgaacctgccacctgcccagctgtcgtcc
agcgatgaggagaccagggaggagctggcccgaattgggttggtgccacc
ccctgaagagtttgccaacggggtcctgctggccaccccactcgctggcc
cgggcccctcgcccaccacggtgcccagcccggcctcagggaagcccagc
agtgagccaccccctgcccctgagtctgcagccgactctggggtggagga
ggctgacacacgcagctccagcgacccccacctggagaccacaagcacca
tctccacggtgtccagcatgtccaccttgagctcggagagcggggaactc
actgacacccacacctccttcgctgacggacacacttttctactcgagaa
gccaccagtgcctcccaagcccaagctcaagtccccgctggggaaggggc
cggtgaccttcagggacccgctgctgaagcagtcctcggacagcgagctc
atggcccagcagcaccacgccgcctctgccgggctggcctctgccgccgg
gcctgcccgccctcgctacctcttccagagaaggtccaagctatgggggg
accccgtggagagccgggggctccctgggcctgaagacgacaaaccaact
gtgatcagtgagctcagctcccgcctgcagcagctgaacaaggacacgcg
ttccctgggggaggaaccagttggtggcctgggcagcctgctggaccctg
ccaagaagtcgcccatcgcagcagctcggctcttcagcagcctcggtgag
ctgagctccatttcagcgcagcgcagccccgggggcccgggcggcggggc
ctcgtactcggtgaggcccagtggccgctaccccgtggcgagacgcgccc
cgagcccggtgaagcccgcgtcgctggagcgggtggaggggctgggggcg
ggcgcggggggcgcagggcggcccttcggcctcacgccccccaccatcct
caagtcgtccagcctctccatcccgcacgagcccaaggaggtgcgcttcg
tggtgcgcagcgtgagcgcgcgcagtcgctccccctcgccgtcgccgctg
ccctcgcccgcgtccggccccggccccggcgcccccggcccacgccgacc
cttccagcagaagccgctgcagctctggagcaagttcgacgtgggcgact
ggctggagagcatccacctaggcgagcaccgcgaccgcttcgaggaccat
gagatagaaggcgcgcacctacccgcgcttaccaaggacgacttcgtgga
gctgggcgtcacgcgcgtgggccaccgcatgaacatcgagcgcgcgctca
ggcagctggacggcagctga

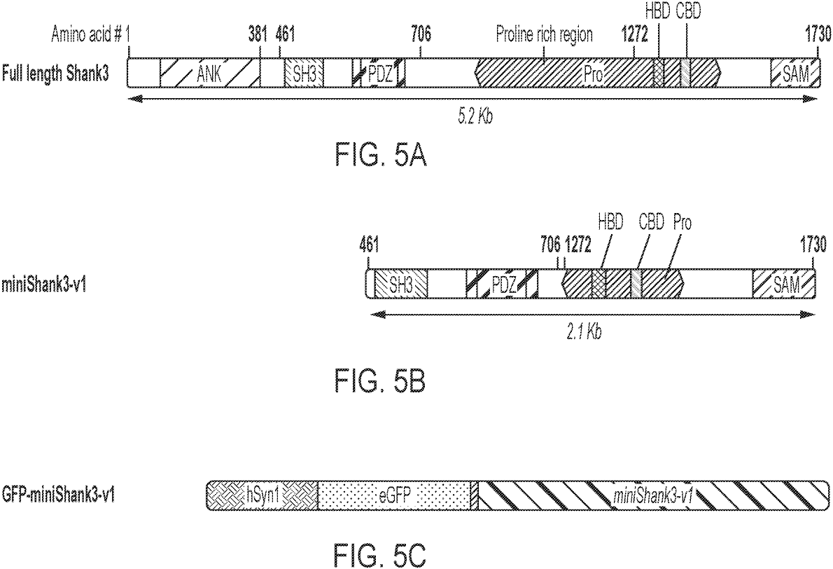

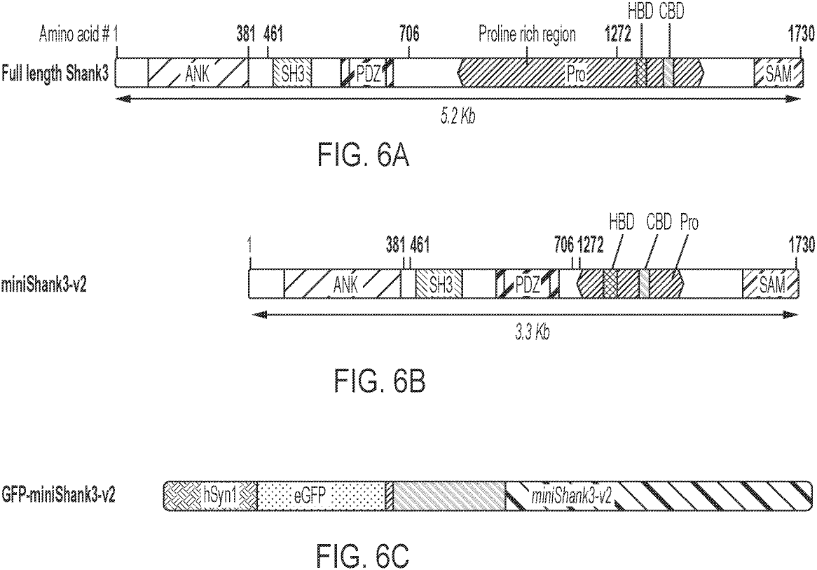

The full-length Shank3 protein comprises multiple domains and is encoded by a gene that is about 5.2 Kb in size. Due to its size, it is difficult to deliver full-length Shank3 to a tissue or cell of interest via an AAV vector. The inventors of the instant application have discovered that specific domains can be removed or truncated from the full-length Shank3 protein to produce MiniShank3 that is efficacious in restoring Shank3 activity in excitatory neurons (FIGS. 5B and 6B). Shank proteins (e.g., Shank3 proteins) encoded by polynucleotides described herein can be miniaturized to form a shortened variant of the native, full length Shank3 protein. As disclosed herein, a miniaturized Shank3 protein, or a DNA construct encoding the miniaturized Shank3 protein, are referred to interchangeably as “miniShank3” or “MiniShank3.”

In some embodiments, the Shank3 protein disclosed herein is expressed as a miniaturized Shank3 DNA construct. In some embodiments, the variant Shank3 DNA constructs and the Shank3 proteins disclosed herein (MiniShank3) comprise fewer domains than the full-length Shank3 gene and protein. In some embodiments, the Shank3 protein disclosed herein is encoded by a non-naturally occurring polynucleotide.

Shank3 proteins encoded by polynucleotides described herein can include one or more protein domains. For example, Shank3 proteins can include one or more of: an SH3 domain, a PDZ domain, a Homer binding domain, a Cortactin domain, a SAM domain, and/or an ankyrin repeat domain.

In some embodiments, the SH3 domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 474-525 of SEQ ID NO: 6 or residues 473-524 of SEQ ID NO: 5. In some embodiments, the SH3 domain comprises at least 90% identity to residues 474-525 of SEQ ID NO: 6. In some embodiments, the SH3 domain comprises at least 90% identity to residues 473-524 of SEQ ID NO: 5. In some embodiments, the SH3 domain comprises residues 474-525 of SEQ ID NO: 6. In some embodiments, the SH3 domain comprises residues 473-524 of SEQ ID NO: 5. In some embodiments, the SH3 domain can comprise any percent identity to residues 474-525 of SEQ ID NO: 6 suitable for construction of the MiniShank3. In some embodiments, the SH3 domain can comprise any percent identity to residues 473-524 of SEQ ID NO: 5 suitable for construction of the MiniShank3.

In some embodiments, the PDZ domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 573-662 of SEQ ID NO: 6 or residues 572-661 of SEQ ID NO: 5. In some embodiments, the PDZ domain comprises at least 90% identity to residues 573-662 of SEQ ID NO: 6. In some embodiments, the PDZ domain comprises at least 90% identity to residues 572-661 of SEQ ID NO: 5. In some embodiments, the PDZ domain comprises residues 573-662 of SEQ ID NO: 6. In some embodiments, the PDZ domain comprises residues 572-661 of SEQ ID NO: 5. In some embodiments, the PDZ domain can comprise any percent identity to residues 573-662 of SEQ ID NO: 6 suitable for construction of the MiniShank3. In some embodiments, the PDZ domain can comprise any percent identity to residues 572-661 of SEQ ID NO: 5 suitable for construction of the MiniShank3.

In some embodiments, the Homer binding domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 1294-1323 of SEQ ID NO: 5 or 6. In some embodiments, the Homer domain comprises at least 90% identity to residues 1294-1323 of SEQ ID NO: 5. In some embodiments, the Homer domain comprises at least 90% identity to residues 1294-1323 of SEQ ID NO: 6. In some embodiments, the Homer domain comprises residues 1294-1323 of SEQ ID NO: 5 or 6. In some embodiments, the Homer domain can comprise any percent identity to residues 1294-1323 of SEQ ID NO: 5 or 6 suitable for construction of the MiniShank3.

In some embodiments, the Cortactin binding domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 1400-1426 of SEQ ID NO: 5 or 6. In some embodiments, the Cortactin binding domain comprises at least 90% identity to residues 1400-1426 of SEQ ID NO: 5. In some embodiments, the Cortactin binding domain comprises at least 90% identity to residues 1400-1426 of SEQ ID NO: 6. In some embodiments, the Cortactin binding domain comprises residues 1400-1426 of SEQ ID NO: 5 or 6. In some embodiments, the Cortactin binding domain can comprise any percent identity to residues 1400-1426 of SEQ ID NOs: 5 or 6 suitable for construction of the MiniShank3.

In some embodiments, the SAM domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 1664-1729 of SEQ ID NO: 6 or to residues 1663-1728 of SEQ ID NO:5. In some embodiments, the SAM binding domain comprises at least 90% identity to residues 1664-1729 of SEQ ID NO: 6. In some embodiments, the SAM binding domain comprises at least 90% identity to residues 1663-1728 of SEQ ID NO: 5. In some embodiments, the SAM domain comprises residues 1664-1729 of SEQ ID NO: 6. In some embodiments, the SAM domain comprises residues 1663-1728 of SEQ ID NO:5. In some embodiments, the SAM binding domain can comprise any percent identity to residues 1664-1729 of SEQ ID NO: 6 suitable for construction of the MiniShank3. In some embodiments, the SAM binding domain can comprise any percent identity to residues 1663-1728 of SEQ ID NO: 5 suitable for construction of the MiniShank3.

In some embodiments, the ankyrin repeat domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 148-345 of SEQ ID NO: 6 or to residues 147-313 of SEQ ID NO: 5. In some embodiments, the ankyrin repeat domain comprises at least 90% identity to residues 148-345 of SEQ ID NO: 6. In some embodiments, the ankyrin repeat domain comprises at least 90% identity to residues 147-313 of SEQ ID NO: 5. In some embodiments, the ankyrin repeat domain can comprise any percent identity to residues 148-345 of SEQ ID NO: 6 suitable for construction of the MiniShank3. In some embodiments, the ankyrin repeat domain can comprise any percent identity to residues 147-313 of SEQ ID NO: 5 suitable for construction of the MiniShank3.

In some embodiments, the MiniShank3 protein is less than 65% identical to SEQ ID NO: 5 over the full length of SEQ ID NO: 5. In some embodiments, the MiniShank3 protein is less than 65% identical to SEQ ID NO: 6 over the full length of SEQ ID NO: 6. As used herein, “less than 65%” includes any percent identity less than 65% that is suitable for construction of the MiniShank3. In some embodiments, the MiniShank3 protein is less than 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11% or 10% identical to SEQ ID NO: 5 over the full length of SEQ ID NO: 5.

In some embodiments, the MiniShank3 protein comprises an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical, or is 100% identical, including all values in between, to any one of SEQ ID NOs: 17-20, provided in Table 1.

In some embodiments, the MiniShank3 protein comprises any one of SEQ ID NOs: 17-20. In some embodiments, SEQ ID NO: 17 is encoded by SEQ ID NO: 1. In some embodiments, SEQ ID NO: 18 is encoded by SEQ ID NO: 2. In some embodiments, SEQ ID NO: 19 is encoded by SEQ ID NO: 3. In some embodiments, SEQ ID NO: 20 is encoded by SEQ ID NO: 4.

In some embodiments, the MiniShank3 protein comprises an ankyrin repeat domain. In certain embodiments in which the MiniShank3 protein comprises an ankyrin repeat domain, the MiniShank3 protein comprises SEQ ID NOs: 19 and/or 20.

In other embodiments, the MiniShank3 protein does not comprise an ankyrin repeat domain. In certain embodiments in which the MiniShank3 protein does not comprise an ankyrin repeat domain, the MiniShank3 protein comprises SEQ ID NOs: 17 and/or 18.

In some embodiments, the sequences of polynucleotides encoding MiniShank3 proteins associated with the disclosure comprise at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between, to any one of SEQ ID NOs: 1-4, and encode one or more proteins with Shank3 activity. In some embodiments, the sequences of polynucleotides encoding MiniShank3 proteins associated with the disclosure comprise at least 90% identity to any one of SEQ ID NOs: 1-4, and encode one or more proteins with Shank3 activity. In some embodiments, the sequences of polynucleotides encoding MiniShank3 proteins associated with the disclosure comprise any one of SEQ ID NOs: 1-4. In some embodiments, any one of SEQ ID NOs: 1-4 encodes one or more proteins with partial Shank3 activity. In some embodiments, any one of SEQ ID NOs: 1-4 encodes one or more proteins with full Shank3 activity.

In some embodiments, the MiniShank3 is encoded by any one of SEQ ID NOs: 1-4, provided in Table 1. SEQ ID NO: 1 and SEQ ID NO: 3 correspond to mouse MiniShank3 nucleic acid sequences, while SEQ ID NO: 2 and SEQ ID NO: 4 correspond to human MiniShank3 nucleic acid sequences. SEQ ID NO: 1 and SEQ ID NO: 2 encode MiniShank3 proteins that do not comprise an ankyrin repeat domain or the N-terminal domain. SEQ ID NO: 3 and SEQ ID NO: 4 encode MiniShank3 proteins that comprise an ankyrin repeat domain and the N-terminal domain.

Polynucleotides described herein that encode MiniShank3 proteins encode proteins that have at least partial Shank3 activity.

As disclosed herein, “identity” of sequences refers to the measurement or calculation of the percent of identical matches between two or more sequences with gap alignments addressed by a mathematical model, algorithm, or computer program that is known to one of ordinary skill in the art. The percent identity of two sequences (e.g., nucleic acid or amino acid sequences) may, for example, be determined using Basic Local Alignment Search Tool (BLAST^{®}) such as NBLAST^{®} and XBLAST^{®} programs (version 2.0). Alignment technique such as Clustal Omega may be used for multiple sequence alignments. Other algorithms or alignment methods may include but are not limited to the Smith-Waterman algorithm, the Needleman-Wunsch algorithm, or Fast Optimal Global Sequence Alignment Algorithm (FOGSAA).

In some embodiments, a polynucleotide encoding the Shank protein as disclosed herein (Shank 1, Shank2, Shank3) is less than about 4.6 kb, about 4.5 kb, about 4.4 kb, about 4.3 kb, about 4.2 kb, about 4.1 kb, about 4.0 kb, about 3.9 kb, about 3.8 kb, about 3.7 kb, about 3.6 kb, about 3.5 kb, about 3.4 kb, about 3.3 kb, about 3.2 kb, about 3.1 kb, about 3.0 kb, about 2.9 kb, about 2.8 kb, about 2.7 kb, about 2.6 kb, about 2.5 kb, about 2.4 kb, about 2.3 kb, about 2.2 kb, or about 2.1 kb in size. In some embodiments, the polynucleotide encoding the Shank protein as disclosed herein (Shank 1, Shank2, Shank3) can be in any size that is suitable for the methods and vectors disclosed in the present disclosure.

### Diseases and Disorders

The present disclosure provides compositions and methods suitable for treating a neurodevelopmental disorder, such as an autism spectrum disorder (ASD), or Phelan-McDermid syndrome.

As used herein “neurodevelopmental disorder” refers to any disorder that impairs the growth and/or development of the brain and/or central nervous system. In some embodiments, neurodevelopmental disorders impact one or more brain functions, such as emotion, learning ability, self-control, and memory. It should be appreciated that aspects of the disclosure may be applicable for treatment of any neurodevelopmental disorder.

In some embodiments, the neurodevelopmental disorder is an autism spectrum disorder (ASD). Diagnosis of ASDs is mainly based on criteria such as deficits in communication, impaired social interaction, and repetitive or restricted interests and behaviors. ASDs are highly heritable disorders with concordance rates as high as 90% for monozygotic twins. However, ASDs are clinically heterogeneous, covering a wide range of discrete disorders of differential symptomatic severity. ASDs are believed to be etiologically heterogeneous, possibly encompassing polygenic, monogenic and environmental factors.

Alterations in synaptic connectivity and function have been proposed as a key mechanism underlying ASDs. Recent genetic studies have identified a large numbers of candidate genes for ASDs, many of which encode synaptic proteins including Shank3, Neuroligin-3, Neuroligin-4 and Neurexin-1. These findings suggest that synaptic dysfunction may underlie a common mechanism for a subset of ASDs. Various Shank3 mutations have been identified as a monogenic cause of ASD with intellectual disability (ID). In ASD patients, all Shank3 deletions and/or mutations that have been identified lead to loss of function (LoF) in one of the two normal copies of the Shank3 gene (i.e. haploinsufficiency). Recent genetic screens also identified a large number of mutations in the Shank3 gene including microdeletions, nonsense mutations and recurrent breakpoints in ASD patients not diagnosed with Phelan-McDermid syndrome (PMS). These implicate Shank3 gene disruption and/or mutation as a monogenic cause of autism spectrum disorder (ASD). The current estimation is that deletions and/or mutations involving Shank3 account for about 2% of all ASD patients with ID. Thus, understanding the function of Shank3 may provide insight into pathological mechanisms of ASD.

As used herein, “intellectual disability” refers to a disability that causes a subject to have deficits in intellectual functioning and/or adaptive functioning. Intellectual functioning can include, for example, reasoning, problem solving, planning, abstract thinking, judgment, academic learning, and/or experiential learning. Intellectual functioning can be measured using any method known in the art, such as by IQ tests. Adaptive functioning can include, for example, skills needed to live in an independent and responsible manner such as communication and social skills. In some instances, intellectual disability can be evident during childhood or adolescence.

In some embodiments, the neurodevelopmental disorder is Phelan-McDermid syndrome (PMS, 22q13.3 deletion syndrome), which is an autism spectrum disorder that shows autistic-like behaviors, hypotonia, severe intellectual disability and impaired development of speech and language. Shank3 is one of the genes that has been reported to be deleted in Phelan-McDermid syndrome. Disruption of Shank3 is thought to be the cause of the core neurodevelopmental and neurobehavioral deficits in Phelan-McDermid syndrome because individuals carrying a ring chromosome 22 with an intact Shank3 gene are phenotypically normal.

Other neurodevelopmental disorders can include but are not limited to attention-deficit/hyperactivity disorder (ADHD), learning disabilities such as dyslexia or dyscalculia, intellectual disability (mental retardation), conduct or motor disorders, cerebral palsy, impairments in vision and hearing, developmental language disorder, neurogenetic disorders such as Fragile X syndrome, Down syndrome, Rett syndrome, hypogonadotropic hypogonadal syndromes, and traumatic brain injury.

### Subjects

A subject to be treated by methods described herein may be a human subject or a non-human subject. Non-human subjects include, for example: non-human primates; farm animals, such as cows, horses, goats, sheep, and pigs; pets, such as dogs and cats; and rodents.

A subject to be treated by methods described herein may be a subject having, suspected of having, or at risk for developing a neurodevelopmental disorder. In some embodiments, a subject has been diagnosed as having a neurodevelopmental disorder, while in other embodiments, a subject has not been diagnosed as having a neurodevelopmental disorder. In some embodiments, the subject is a human subject having, suspected of having, or at risk for developing an autism spectrum disorder (ASD). In some embodiments, the subject is a human subject having, suspected of having, or at risk for developing Phelan-McDermid syndrome. In some embodiments, the subject is a subject having reduced expression of the Shank3 gene relative to a control subject. In some embodiments, the expression of the Shank3 gene is reduced in the subject by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, the control subject is a subject that does not have, is not suspected of having, or is not at risk of having, a neurodevelopmental disorder. In some embodiments, the reduced expression of the Shank3 gene in a subject is caused by disruption of at least one copy of the Shank3 gene. In some embodiments, the disruption of the Shank3 gene comprises a deletion in at least one copy of the Shank3 gene. In some embodiments, the disruption of the Shank3 gene comprises one or more mutations within at least one copy of the Shank3 gene.

In some embodiments, the subject is a human subject who exhibits one or more symptoms of an ASD. In some embodiments, the subject is a human subject who exhibits developmental delay. In some embodiments, the subject is a human subject who exhibits intellectual disability (ID). In some embodiments, the subject is a human subject who exhibits sleep disturbance. In some embodiments, the subject is a human subject who exhibits hypotonia. In some embodiments, the subject is a human subject who exhibits lack of speech. In some embodiments, the subject is a human subject who exhibits language delay. In some embodiments, the subject is a human subject who exhibits any symptoms or signs of an ASD.

In some embodiments, a subject is a human subject who is an adult. In some embodiments, the adult is older than 25 years of age. In some embodiments, the adult is not older than 25 years of age. In some embodiments, the adult is not older than 21 years of age. In some embodiments, the adult is not older than 18 years of age. In some embodiments, the adult is 16 years of age. In some embodiments, a subject is elderly (e.g., 65 years old or older). In some embodiments, the adult can be any age of adulthood that is suitable for the treatment disclosed herein.

In some embodiments, the subject is a human subject who is not an adult. In some embodiments, the human subject is not older than 16 years of age. In some embodiments, the human subject is not older than 10 years of age. In some embodiments, the human subject is 10 years of age or younger. In some embodiments, the human subject is a child or an infant. In some embodiments, the human subject is a toddler. In some embodiments, the human subject is at the fetal stage of development. In some embodiments, the human subject is at the prenatal stage of development.

### Viral Vectors

As disclosed herein, polynucleotides encoding MiniShank3 proteins can be delivered to a tissue or cell of interest in a viral vector. Vectors described herein can be used to deliver a nucleic acid encoding a protein of interest to a subject, including, e.g., delivery to specific organs or to the central nervous system (CNS) of a subject. In some embodiments, the protein of interest is a Shank protein. In some embodiments, the protein of interest is a Shank3 protein. In some embodiments, the protein of interest is a MiniShank3 protein.

In some embodiments, the present disclosure provides a vector comprising a polynucleotide encoding a Shank protein disclosed herein. In some embodiments, the present disclosure provides a vector comprising a polynucleotide encoding a Shank3 protein. In some embodiments, the vector is a viral vector. In some embodiments, the vector is an AAV vector.

AAV refers to a replication-deficient Dependoparvovirus within the Parvoviridae genus of viruses. AAV can be derived from a naturally occurring virus or can be recombinant. AAV can be packaged into capsids, which can be derived from naturally occurring capsid proteins or recombinant capsid proteins. The single-stranded DNA genome of AAV includes inverted terminal repeat (ITRs). ITRs are involved in the replication and encapsidation of the AAV genome, along with its integration in the host genome and its excision. Without wishing to be bound by any theory, AAV vectors can comprise one or more ITRs, including a 5′ ITR and/or a 3′ ITR, one or more promoters, one or more nucleic acid sequences encoding one or more proteins of interest, and/or additional posttranscriptional regulator elements. AAV vectors disclosed herein can be prepared using standard molecular biology techniques known to one of ordinary skill in the art, as described, for example, in Sambrook el al. (Molecular Cloning: A Laboratory Manual. Cold Spring Harbor Laboratory Press, N.Y. (2012)), which is incorporated herein by reference in its entirety.

In some embodiments, AAV integrates into a host cell genome. In some embodiments, AAV does not integrate into a host genome. In some embodiments, AAV vectors disclosed herein can include sequences from any known organism. In some embodiments, AAV vectors disclosed herein can include synthetic sequences. AAV vector sequences can be modified in any way known to one of ordinary skill in the art, such as by incorporating insertions, deletions or substitutions, and/or through the use of posttranscriptional regulatory elements, such as promoters, enhancers, and transcription and translation terminators, such as polyadenylation signals. In some embodiments, AAV vectors can include sequences related to replication and integration.

In some embodiments, a MiniShank3 as disclosed herein is delivered to a tissue or a cell of interest via an AAV vector. In some embodiments, the AAV vector delivering the MiniShank3 as disclosed herein is delivered to the central nervous system (CNS) of a subject. As used herein, delivering the AAV vector to the CNS may include delivering the AAV vector to any tissue or cell of interest in the CNS. In some embodiments, delivering the AAV vector to the CNS involves delivering the AAV vector to neuronal tissues or cells. In some embodiments, delivering the AAV vector to the CNS involves delivering the AAV vector to the brain. In some embodiments, delivering the AAV vector to the CNS involves delivering the AAV vector to the spinal cord. In some embodiments, delivering the AAV vector to the CNS involves delivering the AAV vector to the white and gray matter. In some embodiments, the AAV vector delivering the MiniShank3 as disclosed herein is delivered to any tissue or cell of interest of a subject that is suitable for the treatments as disclosed herein.

As used in the present disclosure, “delivering” or “administering” an AAV vector can include any method known in the art for delivering or administering an AAV vector or a composition comprising an AAV vector to a subject. Administering can include but is not limited to direct administration of an AAV vector or a composition comprising the AAV vector, or peripheral administration via passive diffusion or convection-enhanced delivery (CED) to bypass the blood brain barrier as known in the art. AAV vectors described herein can be administered in any composition that would be compatible with aspects of the disclosure.

AAV vectors can include any known AAV serotype, including, for example, AAV1, AAV2, AAV3, AAV4, AAV5, AAV6, AAV7, AAV8, AAV9, AAV10, and AAV11. In some embodiments, the AAV serotype is AAV9. Clades of AAV viruses are described in, and incorporated by reference, from Gao et al. (2004) J. Virol. 78(12):6381-6388. In some embodiments, any AAV serotype that is suitable for delivery to the CNS may be selected.

AAV vectors of the present disclosure may comprise or be derived from any natural or recombinant AAV serotype. In some embodiments, the AAV vector may utilize or be based on an AAV serotype described in WO 2017/201258A1, the contents of which are incorporated herein by reference in its entirety, such as, but not limited to, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43- 23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2- 3/rh.61, AAV2-4/rh.50, AAV2-5/rh.51, AAV3.1/hu.6, AAV3.1/hu.9, AAV3-9/rh.52, AAV3- 11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV- PAEC6, AAV-PAEC7, AAV-PAEC8, AAV-PAEC11, AAV-PAEC12, AAV-2-pre-miRNA- 101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.⅟hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr- E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B 1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1- 1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp- 8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B (PHP.B), AAV-PHP.A (PHP.A), G2B-26, G2B-13, TH1.1-32 and/or TH1.1-35, and variants thereof. AAV vectors are described further in U.S. 9,585,971, U.S. 2017/0166926, and WO2020/160337, which are incorporated by reference herein in their entireties.

In some embodiments, a MiniShank3 disclosed herein is delivered by an AAV vector. In some embodiments, the AAV vector comprises a transgene and its regulatory sequences, and optionally 5′ and 3′ ITRs. In some embodiments, the transgene and its regulatory sequences are flanked by the 5′ and 3′ ITR sequences. The transgene may comprise, as disclosed herein, one or more regions that encode a MiniShank3. The transgene may also comprise a region encoding for another protein. The transgene may also comprise one or more expression control sequences (e.g., a poly-A tail). In some embodiments, an AAV vector comprises at least AAV ITRs and a MiniShank3 transgene.

In some embodiments, the AAV may be packaged into an AAV particle and administered to a subject and/or delivered to a selected target cell. In some embodiments, the AAV particle comprises an AAV capsid protein. In some embodiments, the AAV particle comprises at least one capsid protein that is selected from the AAV serotypes as disclosed herein including AAV1, AAV2, AAV3, AAV4, AAV5, AAV6, AAV6.2, AAV7, AAV8, AAV9, PHB.eB, AAV.rh8, AAV.rh10, AAV.rh39, AAV.43, AAV2/2-66, AAV2/2-84, and AAV2/2-125, or a variant of any of the foregoing.

In some embodiments, the miniShank3 transgene coding sequence in the AAV vector is operably linked to regulatory sequences for tissue-specific gene expression. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the tissue-specific regulatory sequence can be a Syn promoter (e.g., hSyn1). In some embodiments, the tissue-specific regulatory sequence can be any promoter or enhancer that is neuron-specific and is suitable for the treatments described herein.

In some embodiments, a miniShank3 transgene coding sequence comprising SEQ ID NO: 2 or 4 in an AAV vector is operably linked to a promoter and is flanked by AAV ITRs. In some embodiments, a miniShank3 transgene coding sequence comprising SEQ ID NO: 1 or 3 in an AAV vector is operably linked to a promoter and is flanked by AAV ITRs.

Aspects of the disclosure relate to AAV vectors expressing miniShank3 transgenes. In some embodiments, a miniShank3 transgene is flanked by AAV ITRs. In some embodiments, the AAV ITRs comprise AAV2 ITRs. In some embodiments, the AAV ITRs comprise AAV1 ITRs. In some embodiments, the AAV ITRs comprise AAV5 ITRs. In some embodiments, the AAV ITRs comprise AAV6 ITRs. In some embodiments, the AAV ITRs comprise AAV8 ITRs. In some embodiments, the AAV ITRs comprise AAV9 ITRs. In some embodiments, the AAV ITRs comprise rh10 ITRs. In some embodiments, the AAV ITRs may include self-complementary ITRs.

It should be appreciated that AAV vectors described herein can include DNA constructs that comprise a transgene such as MiniShank3, 5′ and/or 3′ ITRs, promoters, introns, and/or other associated regulatory elements that are known in the art.

In some embodiments, the AAV vector comprises a Woodchuck Hepatitis Virus Posttranscriptional Regulatory Element (WPRE), which may enhance miniShank3 transgene expression. In some embodiments, the AAV vector comprises an untranslated portion such as an intron or a 5′ or 3′ untranslated region. In some embodiments, the intron may be located between the promoter/enhancer sequence and the miniShank3 transgene.

In some embodiments, the AAV vector used herein may be a self-complementary vector.

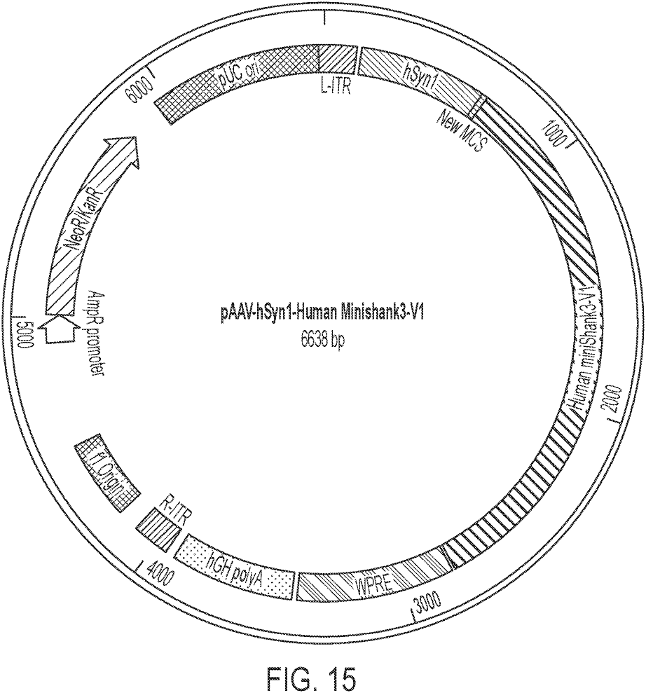

FIG. 15 shows an example of an AAV vector (referred to in FIG. 15 as “AAV-hSyn1-HumanMiniShank3-V1”) comprising a human miniShank3 transgene expressed under the control of the human synapsin 1 (hSyn1) promoter. The AAV vector shown in FIG. 15 comprises a sequence provided as SEQ ID NO: 21 in Table 1.

As shown in FIG. 15, SEQ ID NO: 21 comprises a human Mini-Shank3 gene, a 5′-ITR, a 3′-ITR, a WPRE, an hGH polyA, an F1 origin, a NeoR/KanR marker, a hSyn1 promoter, and a PUC origin. In some embodiments, the Inverted terminal repeat (ITR) sequences comprise about 145 nucleotides each. These elements may be useful in cis for effective replication and encapsidation. A skilled person in the art would appreciate that any elements of AAV vectors known in the art may be compatible with aspects of the disclosure. One of skill in the art would also appreciate that any of the polynucleotide sequences described herein that encode a functional MiniShank3 protein can be expressed in a DNA construct similar to that shown in FIG. 15 for AAV delivery. These DNA constructs may include one or more of the elements shown in FIG. 15. For example, in some embodiments a coding sequence comprising at least 70%, at least 75%, at least 80%, at least 85%, at least 90%, or at least 95% identity to any one of SEQ ID NOs: 1-4 is expressed in a DNA construct such as that shown in FIG. 15. In some embodiments a coding sequence comprising the sequence of any one of SEQ ID NOs: 1-4 is expressed in a DNA construct such as that shown in FIG. 15. In some embodiments, the DNA construct includes one or more of the elements shown in FIG. 15, such as a promoter, a 5′-ITR, a 3′-ITR, a WPRE, an hGH polyA, an F1 origin, a NeoR/KanR marker, and/or a PUC origin.

In some embodiments, an AAV vector associated with the disclosure includes a nucleic acid sequence encoding a MiniShank3 protein that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to the sequence of SEQ ID NO: 21.In some embodiments, an AAV vector comprises a sequence corresponding to SEQ ID NO: 21, which encodes a MiniShank3 protein comprising the sequence of SEQ ID NO: 18.

In some embodiments, an AAV vector that includes a sequence that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to the sequence of SEQ ID NO: 21, and which encodes a MiniShank3 protein comprising the sequence of SEQ ID NO: 18, may be delivered to a human subject in need thereof and may be suitable for treating a human subject who has a neurodevelopmental disorder.

As one of ordinary skill in the art would appreciate, any method known in the art for designing AAV vectors for clinical use, and for delivery of AAV vectors, may be compatible with aspects of the disclosure. For example, non-limiting examples of disclosure related to AAV vectors and delivery are provided in and incorporated by reference from U.S. Pat. No. 7,906,111, entitled “Adeno-associated virus (AAV) clades, sequences, vectors containing same, and uses therefor” and U.S. Pat. No. 9,834,788, entitled “AAV -vectors for use in gene therapy of choroideremia,” each of which is incorporated by reference herein in its entirety.

In some embodiments, an AAV vector associated with the disclosure includes a sequence encoding a MiniShank3 protein for AAV delivery that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to the sequence of SEQ ID NO: 7 or 21, provided in Table 1. SEQ ID NO: 7 encodes the protein sequence of SEQ ID NO: 11. SEQ ID NO: 8 encodes the protein sequence of SEQ ID NO: 12. SEQ ID NO: 9 encodes the protein sequence of SEQ ID NO: 13. SEQ ID NO: 10 encodes the protein sequence of SEQ ID NO: 14. SEQ ID NO: 21 encodes the protein sequence of SEQ ID NO: 18.

In some embodiments, the AAV vector encoding a MiniShank3 protein for AAV delivery encodes a protein with a sequence that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, to any one of SEQ ID NOs: 11 or 17-20, provided in Table 1.

In some embodiments, the vector used for delivering the miniShank3 as disclosed herein can be a lentivirus vector. In some embodiments, the vector used for delivering the miniShank3 as disclosed herein can be an adenovirus vector.

In some embodiments, the vector construct disclosed herein can comprise SEQ ID NO: 21 as shown in Table 1.

In some embodiments, the vector comprising the polynucleotide of the Shank3 protein (i.e., the miniShank3 DNA construct) can be expressed in a specific tissue or cell of interest. In some embodiments, the vector disclosed herein comprises a promoter. In some embodiments, the vector comprises a cell-type specific promoter. In some embodiments, the promoter is a human promotor. In some embodiments, the human promoter is human Synapsin 1 (hSyn1). In some embodiments, the hSynlpromotor has a polynucleotide sequence corresponding to SEQ ID NO: 22. In some embodiments, the human promoter can be any promotor that is known in the art and is suitable for construction of the miniShank3. In some embodiments, the human promoter can be any promoter that has high specificity for neuronal tissues and cells. In some embodiments, the promoter can be a constitutive promoter. For example, the constitutive promoter can be a CAG promoter. As one of ordinary skill in the art would appreciate, any promoter may be used so long as the selected promoter is compatible with aspects of the disclosure.

### Compositions and Administration

The present disclosure provides compositions, including pharmaceutical compositions, comprising a polynucleotide (e.g., miniShank3) delivered in an AAV vector as disclosed herein and a pharmaceutically acceptable carrier.

The compositions of the disclosure may comprise an AAV alone, or in combination with one or more other viruses. In some embodiments, a composition comprises 1, 2, 3, 4, 5, 6, 7, 8, 9, 10, or more different AAVs each having one or more different Shank protein.

Suitable carriers may be readily selected by one of ordinary skill in the art in view of the indication for which the AAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure. Pharmaceutical compositions comprising AAV vectors are described further in U.S. 9,585,971 and U.S. 2017/0166926, which are incorporated by reference herein in their entireties.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the AAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the AAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 µm. Sonication of MLVs results in the formation of small unilamellar vesicles (SUVs) with diameters in the range of 200 to 500 Å, containing an aqueous solution in the core.

Alternatively, nanocapsule formulations of the AAV vector may be used. Nanocapsules can generally entrap substances in a stable and reproducible way. To avoid side effects due to intracellular polymeric overloading, such ultrafine particles (sized around 0.1 µm) should be designed using polymers able to be degraded in vivo. Biodegradable polyalkyl-cyanoacrylate nanoparticles that meet these requirements are contemplated for use.

In some embodiments, the pharmaceutical composition comprising a nucleic acid delivered in an AAV vector comprises other pharmaceutical ingredients, such as preservatives, or chemical stabilizers. Suitable exemplary preservatives include chlorobutanol, potassium sorbate, sorbic acid, sulfur dioxide, propyl gallate, parabens, ethyl vanillin, glycerin, phenol, thimerosal, and parachlorophenol. Suitable chemical stabilizers include gelatin and albumin. In many cases, it will be preferable to include isotonic agents, for example, sugars or sodium chloride. Prolonged absorption of the pharmaceutical compositions can be brought about by the use in the compositions of agents delaying absorption, for example, aluminum monostearate and gelatin.

The pharmaceutical forms suitable for delivering the AAV vectors include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi.

Methods described herein comprise administering AAV vector in sufficient amounts to transfect the cells of a desired tissue (e.g., brain) and to provide sufficient levels of gene transfer and expression without undue adverse effects. Conventional and pharmaceutically acceptable routes of administration include, but are not limited to, direct delivery to the selected organ, oral, inhalation, intraocular, intravenous including facial vein injection and retroorbital injection, intracerebroventricular (ICV), intramuscular, intrathecal, intracranial, subcutaneous, intradermal, intratumoral, and other parental routes of administration. Routes of administration may be combined, if desired. In some embodiments, the vector as disclosed herein is administered intravenously.

In some embodiments, the present disclosure provides methods of treating a subject having a neurodevelopmental disorder. In some embodiments, the present disclosure provides methods of treating a subject having an autism spectrum disorder (ASD). In some embodiments, the present disclosure provides methods of treating a subject having Phelan-McDermid syndrome. Methods provided herein, in some embodiments, comprise administering and delivering an effective amount of a composition comprising a vector that comprises a polynucleotide encoding a Shank3 protein (e.g., miniShank3) to a target environment or tissue of a subject. In some embodiments, the target tissue is cortex. In some embodiments, the target tissue is striatum. In some embodiments, the target tissue is thalamus cerebellum. In some embodiments, the target tissue is hippocampus. In some embodiments, the target tissue is any brain structure. In some embodiments, methods for administering and delivering an effective amount of a composition comprising a vector that comprises a polynucleotide encoding a Shank3 protein (e.g., miniShank3) to a target environment or tissue comprise delivering the composition to neurons or other brain cell types. In some embodiment, the vector is an AAV vector. In some embodiments, methods for delivering a nucleic acid to a target environment or tissue of a subject in need thereof comprise providing a composition comprising an AAV vector comprising at least a nucleic acid (e.g., miniShank3) to be delivered to the target environment or tissue of the subject and administering the composition to the subject. Methods of use of AAV vectors are described further in U.S. 9,585,971, U.S. 2017/0166926, and WO2020/160337, which are incorporated by reference herein in their entireties. In some embodiments, the composition may comprise a capsid protein.

In some embodiments, the composition comprising a vector that comprises a polynucleotide encoding a Shank3 protein is delivered to the subject via intravenous administration, systemic administration, intracerebroventricular administration, in utero administration, intrathecal administration, retro-orbital injection, or facial vein injection. In some embodiments, in utero administration is used for a subject who is at the prenatal stage of development. In some embodiments, the composition is delivered to a subject via a nanoparticle. In some embodiments, the composition is delivered to a subject via a viral vector. In some embodiments, the composition is delivered to a subject via any carriers suitable for delivering nucleic acid materials.

Any composition comprising a vector that comprises a polynucleotide encoding a protein that would be of some use or benefit to the subject may be delivered to a target environment or tissue of the subject according to methods disclosed herein

In addition to the methods of delivery described above, the following techniques are also contemplated as alternative methods of delivering the AAV compositions to a host. Sonophoresis (ie., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

The dose of AAV comprising a polynucleotide that encodes a Shank3 protein (e.g., miniShank3) required to achieve a particular “therapeutic effect,” e.g., the units of dose in absolute vector genomes (vg) or vector genomes per milliliter of pharmaceutical solution (vg/mL) will vary based on several factors including, but not limited to: the route of AAV administration, the level of gene expression required to achieve a therapeutic effect, the specific disease or disorder being treated, and the stability of the gene product. Doses that give maximal percentage of infection without affecting neurodevelopment are also suitable. One of skill in the art can readily determine a AAV dose range to treat a patient having a particular disease or disorder based on the aforementioned factors, as well as other factors.

An effective amount of AAV vector is an amount sufficient to infect an animal or human subject or target a desired tissue. The effective amount will depend primarily on factors such as the species, age, gender, weight, health of the subject, and the tissue to be targeted, and may thus vary among subjects and tissues. The term “effective amount” or “amount effective” in the context of a composition or dose for administration to a subject refers to an amount of the composition or dose that produces one or more desired responses in the subject. In some embodiments, an effective amount of a composition disclosed herein may partially or fully rescue the effects of a mutated Shank3 gene and/or partially or fully restore loss of function of the Shank3 protein. An effective amount can involve reducing the level of an undesired response, although in some embodiments, it involves preventing an undesired response altogether. An effective amount can also involve delaying the occurrence of an undesired response. An effective amount can also be an amount that produces a desired therapeutic endpoint or a desired therapeutic result. In other embodiments, the amounts effective can involve enhancing the level of a desired response, such as a therapeutic endpoint or result. The achievement of any of the foregoing can be monitored by routine methods and the methods as disclosed in the present application. Effective amounts will depend, of course, on the particular subject being treated; the severity of a condition; the individual patient parameters including age, physical condition, size and weight; the duration of the treatment; the nature of concurrent therapy (if any); the specific route of administration and like factors.

For example, in some embodiments, the number of vector genomes administered to the subject is any value between about 6.0×10¹¹ vg and about 9.0×10¹³ vg. In some embodiments, the number of vector genomes administered to the subject is any value between about 6.0 ×10¹³ vg/mL and about 9.0 ×10¹³ vg. In some embodiments, the number of vector genomes administered to the subject is any value between about 1×10¹⁰ to about 1×10¹² vg. In certain embodiments, the effective amount of AAV is 10¹⁰, 10¹¹, 10¹², 10¹³, or 10¹⁴ genome copies per kg. In certain embodiments, the effective amount of AAV is 10¹⁰, 10¹¹, 10¹², 10¹³, 10¹⁴, or 10¹⁵ genome copies per subject. In some cases, a dosage between about 10¹¹ to 10¹³ AAV genome copies is appropriate. In some embodiments, the number of vector genomes administered to the subject can be any dose that is suitable for the treatments and methods disclosed herein.

In some embodiments, a dose of AAV is administered to a subject no more than once per calendar day (e.g., a 24-hour period). In some embodiments, a dose of AAV is administered to a subject no more than once per 2, 3, 4, 5, 6, or 7 calendar days. In some embodiments, a dose of AAV is administered to a subject no more than once per calendar week (e.g., 7 calendar days). In some embodiments, a dose of AAV is administered to a subject no more than bi-weekly (e.g., once in a two calendar week period). In some embodiments, a dose of AAV is administered to a subject no more than once per calendar month (e.g., once in 30 calendar days). In some embodiments, a dose of AAV is administered to a subject no more than once per six calendar months. In some embodiments, a dose of AAV is administered to a subject no more than once per calendar year (e.g., 365 days or 366 days in a leap year). In some embodiments, a dose of rAA V is administered to a subject no more than once per two calendar years (e.g., 730 days or 731 days in a leap year). In some embodiments, a dose of AAV is administered to a subject no more than once per three calendar years (e.g., 1095 days or 1096 days in a leap year).

Formulation of pharmaceutically-acceptable excipients and carrier solutions disclosed herein is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Expression of Proteins Associated With the Shank Protein Network

Methods and compositions provided herein, in some embodiments, are useful for treating a neurodevelopmental disorder, such as, for example, an autism spectrum disorder (ASD), or Phelan-McDermid syndrome. The inventors of the present disclosure discovered that delivery of a miniShank3 via a viral vector such as AAV vector in a mouse model is effective in restoring functionality of postsynaptic density (PSD) proteins. In some embodiments, expression levels of PSD proteins are used to evaluate the efficacy of the administration of the miniShank3. In some embodiments, the PSD protein is Homer. In some embodiments, the PSD protein is post-synaptic density protein 95 (PSD95). In some embodiments, the PSD protein is SynGap1. In some embodiments, the PSD protein is SAPAP3. In some embodiments, the PSD protein is NR1. In some embodiments, the PSD protein is NR2B. In some embodiments, the PSD protein is GluR2. In some embodiments, the PSD protein is any protein that can be improved or restored upon the miniShank3 treatment.

In some embodiments, an increase of any of the PSD proteins, as compared to an untreated control subject, may indicate efficacy of the miniShank3. Methods for detecting gene expression and protein levels are well-known in the art.

In some embodiments, expression of Homer in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of post-synaptic protein (PSD95) in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of SynGap1 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of SAPAP3 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of NR1 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of NR2B in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of GluR2 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administration of a MiniShank3 or a composition comprising a MiniShank3 can lead to improving sleep efficiency. In some embodiments, a subject has improved sleep efficiency after being administered an effective amount of a composition comprising an expression construct comprising a polynucleotide encoding a Shank protein such as a MiniShank3 protein. In some embodiments, the sleep efficiency in the subject after being administered to an effective amount of the composition is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Improved sleep efficiency includes less sleep disturbance, which includes but is not limited to having trouble falling and staying asleep. Measurement of sleep efficiency can be conducted using any methods known in the art.

In some embodiments, administration of a MiniShank3 or a composition comprising a MiniShank3 can lead to improving social impairment. In some embodiments, the social impairment of the subject is improved after being administered an effective amount of a composition comprising an expression construct comprising a polynucleotide encoding a Shank protein such as a MiniShank3 protein. In some embodiments, the social impairment in the subject after being administered to an effective amount of the composition is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Measurement of social impairment can be conducted using any methods known in the art.

As used herein, “social impairment” refers to behavioral abnormalities or defects that prohibit a subject from displaying voluntary social interaction.

In some embodiments, administration of a MiniShank3 or a composition comprising a MiniShank3 can lead to improving locomotion and/or motor coordination deficits. In some embodiments, the locomotion and/or motor coordination deficits of the subject are improved after being administered an effective amount of a composition comprising an expression construct comprising a polynucleotide encoding a Shank protein such as a MiniShank3 protein. In some embodiments, the locomotion and/or motor coordination deficits in the subject after being administered to an effective amount of the composition is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Measurement of locomotion and/or motor coordination deficits can be conducted using any methods known in the art.

As used herein, “locomotion and/or motor coordination deficits” can include, for example, lack of coordination, loss of balance, and/or a shuffling gait.

In some embodiments, administration of a MiniShank3 or a composition comprising a MiniShank3 can lead to improvement in cortical-striatal synaptic dysfunction. In some embodiments, the cortical-striatal synaptic dysfunction of the subject are improved after being administered an effective amount of a composition comprising an expression construct comprising a polynucleotide encoding a Shank protein such as a MiniShank3 protein. In some embodiments, the cortical-striatal synaptic dysfunction in the subject after being administered to an effective amount of the composition is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Measurement of cortical-striatal synaptic dysfunction can be conducted using any methods known in the art.

As used herein, “cortical-striatal synaptic dysfunction” refers to defective corticostriatal circuits in the brain that can cause repetitive and compulsive behaviors, such as in neuropsychiatric disorders and neurodevelopmental diseases such as autism, obsessive-compulsive disorders, and Tourette syndrome.

Some aspects of the technology described herein may be understood further based on the non-limiting illustrative embodiments described in the below Examples section. Any limitations of the embodiments described in the below Examples section are limitations only of the embodiments described in the below Examples section and are not limitations of any other embodiments described herein.

### EXAMPLES

In order that the invention described herein may be more fully understood, the following examples are set forth. The examples described in this application are offered to illustrate the systems and methods provided herein and are not to be construed in any way as limiting their scope.

### Example 1: Mouse Models With Shank3 Mutations Exhibited Synaptic Defects and Behavioral Abnormalities

Multiple mouse and monkey models with Shank3 LoF mutations were developed previously to study how mutations of the Shank3 gene affect brain development, neuronal structure, synaptic and circuit function, and behavior. These models serve as the basis for testing potential therapeutics.

Two different alleles of Shank3 mutant mice were generated: Shank3A and Shank3B. In Shank3A mutant mice, a portion of the gene encoding the ankyrin repeats was targeted, resulting in a complete elimination of Shank3α, the longest Shank3 isoform. However, the other two isoforms were not affected (here named Shank3_{β} and Shank3_{γ}). In Shank3B mutants, the PDZ domain was targeted, leading to complete elimination of both Shank3_{α} and Shank3_{β} isoforms and a significant reduction of the putative Shank3_{γ} isoform. Further analysis was mainly focused on the Shank3B mutant mice.

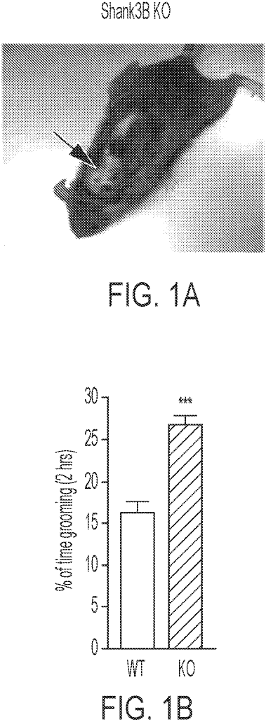

Shank3B^{-/-} mice did not display gross brain abnormalities via histological analysis. However, by the age of 3-6 months, Shank3B^{-/-} mice developed pronounced skin lesions. The lesions were self-inflicted, as they were present in animals isolated at weaning age, and not due to excessive allogrooming, as no lesions were found in wildtype (WT) mice housed from birth with Shank3B^{-/-} mice. Twenty-four hour videotaping revealed that pre-lesion Shank3B^{-/-} mice showed an increase in time spent grooming when compared to WT controls (FIGS. 1A-1B), indicating that Shank3B^{-/-} mice had excessive grooming and self-injurious behavior. In conclusion, Shank3B mutant mice exhibited repetitive and compulsive grooming leading to skin lesions.

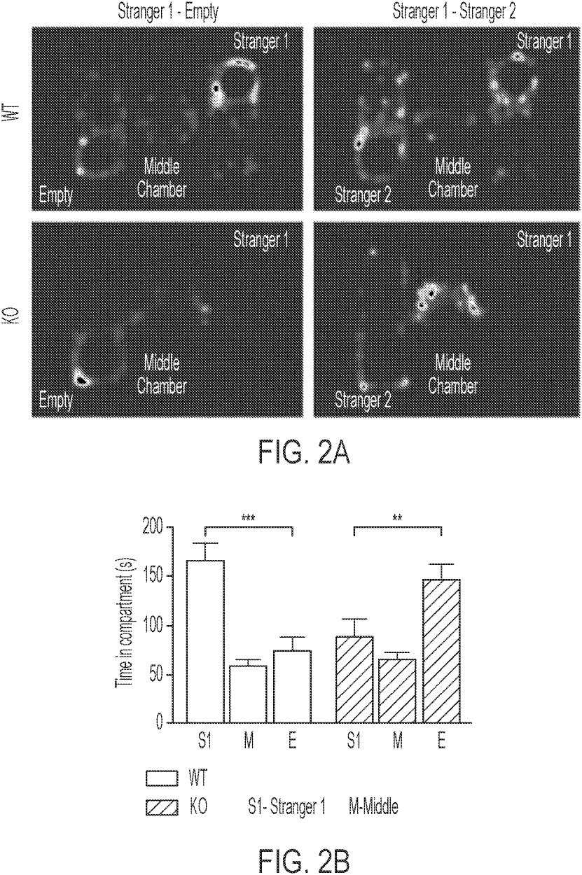

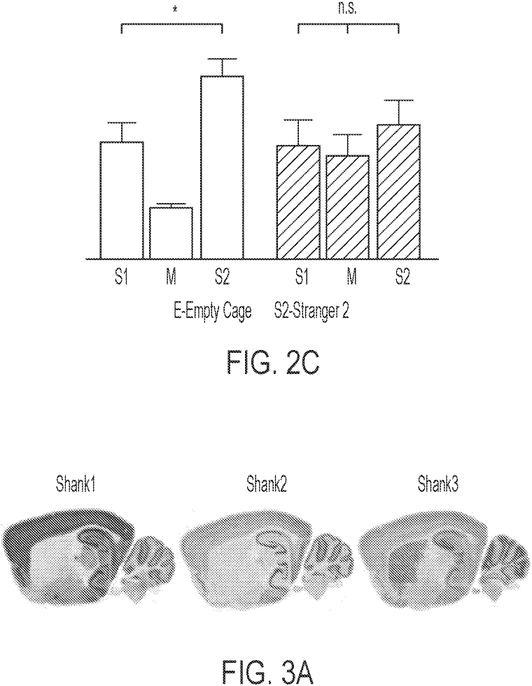

To study the effects of Shank3 mutants on social behaviors, a three-chamber social arena was used to probe animals for their voluntary initiation of social interaction and their ability to discriminate social novelty. Initially, the test animal was left to explore and initiate social contact with a partner (“Stranger 1”) held inside a wired cage or an identical but empty wired cage (“Empty Cage”). The Shank3B^{-/-} mice exhibited a clear preference for interacting with the empty cage rather than with the social partner (FIGS. 2A-2B). In a subsequent trial, a novel social partner (“Stranger 2”) was introduced into the previously empty wired cage. WT mice displayed a preference for the novel animal, whereas Shank3B^{-/-} mutants spent more time in the center chamber (FIGS. 2A and 2C). In conclusion, Shank3B mutant mice displayed social interaction deficits.

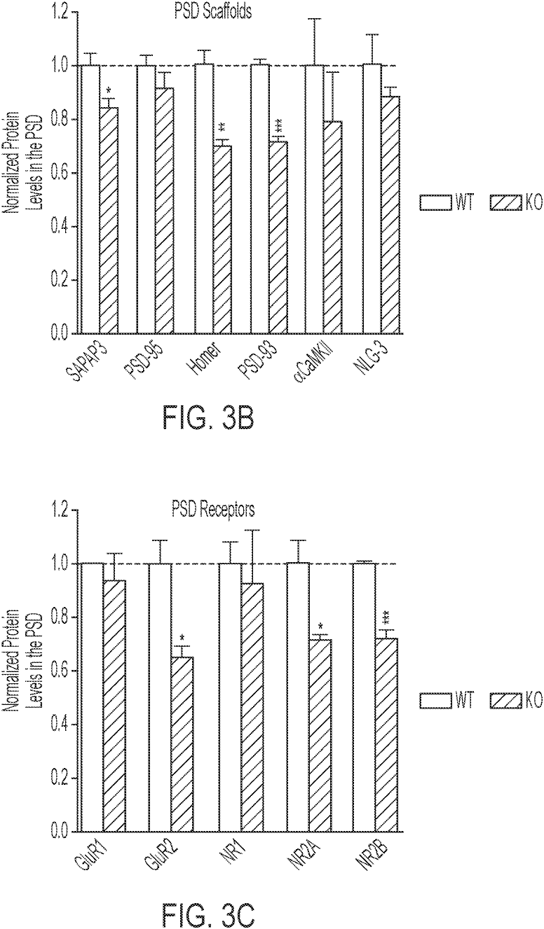

The Shank3 mutant mice were used for studying the effects of Shank3 mutant on striatal synapses. The basal ganglia are one of the brain regions implicated in ASD. The repetitive grooming behavior in Shank3B^{-/-} mice suggested defects in cortico-striatal function. Furthermore, Shank3 was the only Shank family member highly expressed in the striatum (FIG. 3A). Thus, the analyses were focused on striatal neurons and cortico-striatal synapses.

To determine how the disruption of Shank3 may affect the PSD protein network, purified PSDs from the striatum were probed for scaffolding proteins and glutamate receptor subunits (FIGS. 3B-3C). Reduced levels of SAPAP3, Homer-1b/c and PSD93, and glutamate receptor subunits GluR2, NR2A and NR2B in Shank3B^{-/-} mice were observed. This suggested an altered molecular composition of the PSD in the striatum and a disruption of glutamatergic signaling, supporting the hypothesis of Shank proteins being a master scaffold.

Using Golgi tracing, neuronal hypertrophy was found, as shown by an increase in complexity of dendritic arbors, total dendritic length and surface area in Shank3B⁻ ^{/-} MSNs. In addition, dye filing of MSNs revealed a reduction of spine density in Shank3B^{-/-} mice. EM analysis showed a reduction in mean thickness and length of PSDs from Shank3B^{-/-} mice relative to WT. Taken together, these results highlighted a critical role for Shank3 in the development of MSNs and glutamatergic synapses in the striatum.

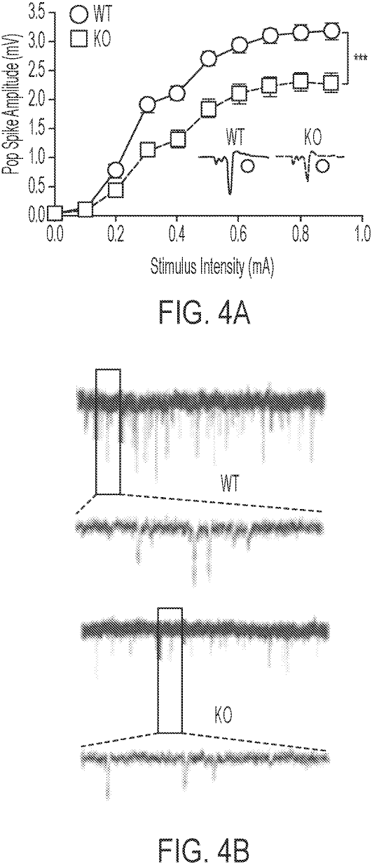

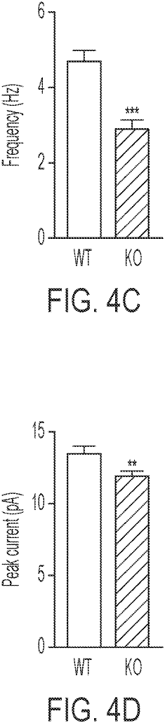

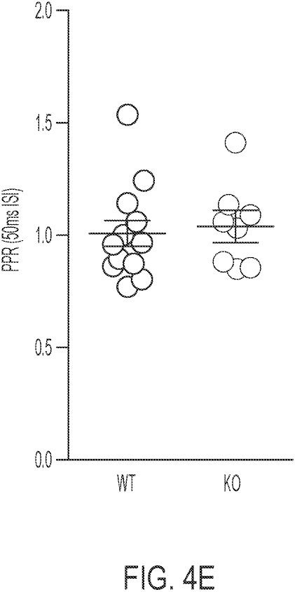

To elucidate the functional consequences of a disruption in Shank3 on synapses, recordings of cortico-striatal synaptic circuitry were performed in acute brain slices of 6-7 week old Shank3B^{-/-} mice, the same age behavioral tests were performed. It was found that field population spikes were significantly reduced in Shank3B^{-/-} mice when compared with controls (FIG. 4A). Presynaptic function was not obviously altered, as indicated by the relationship of stimulation intensity to the amplitude of the action potential component of the response termed negative peak 1 (NP1) and the paired-pulse ratio (PPR). These results indicated that the reduction in total field responses was most likely due to a postsynaptic impairment in synaptic function and/or a reduction in the number of functional synapses.

Whole-cell voltage clamp recordings of AMPAR-mEPSCs in dorsolateral striatal MSNs were also performed. The frequency of mEPSCs was significantly reduced in Shank3B^{-/-} MSNs (FIGS. 4B-4C), suggesting a reduction in the number of functional synapses in Shank3B^{-/-} MSNs since defects on PPR were not observed (FIG. 4E). The peak mEPSC amplitude in Shank3B^{-/-} MSNs was also reduced (FIGS. 4B and 4D), indicating a reduction in the postsynaptic response from the available synapses. These data demonstrated a critical role for Shank3 in postsynaptic function in cortico-striatal circuitry.

Similar examinations will be tested on monkey models and then on human ASD patients with Shank3 mutations in clinical trials.

### Example 2: Design and Construction of miniShank3 Genes

The gene therapy strategy for Shank3 mutations as disclosed herein was to use AAV to deliver a functional copy of Shank3 cDNA into the brain cells to restore the level of Shank3 expression in patients. However, Shank3 is a large protein and the coding sequence is ~5.7kb, exceeding the packaging capacity of AAV vectors. To solve this problem, a miniaturized Shank3 (miniShank3) was designed with the goal of reducing the size while keeping its function intact.

In the first version of miniShank3 (miniShank3-v1, FIG. 5B), the N-terminal domain and other regions such as the Ankyrin repeats and the link sequence between PDZ domain and the Proline-rich domain including a major portion of the Proline-rich domain that were deemed not critical were deleted. This effectively reduced Shank3 coding sequences to 2.1 kb by maintaining only domains predicted to be critical to function of the Shank3 protein.

Without wishing to be bound by any theory, the N-terminal domain (NTD) of the Shank3 protein may play a specialized role in synaptic plasticity. Synaptic plasticity refers to the ability of a neuron to modulate its synaptic strength in response to various stimuli, which is believed to be the cornerstone of a human’s capacity to learn and to adapt to environmental changes. Shank3 has been found to have a specific interaction with a major synaptic plasticity regulator, CaMKIIα. A missense mutation identified in a human ASD patient with severe ID impairs this interaction. A knock-in mouse carrying the same mutation was generated and these mice had synaptic plasticity defects. Based on these findings, it was speculated that adding the NTD to miniShank3-v1 could further improve the function of miniShank3.

A second version of miniShank3 was designed that contained the NTD (miniShank3-v2, FIG. 6B). The miniShank3-v2 (3.1 kb) was significantly larger than the miniShank3-v1 (2.1 kb), but still within the AAV packaging capacity. Based on miniShank3-v1 data, it was expected that both versions would be effective in restoring Shank3 function. The miniShank3-v2 may have certain advantages in restoring synaptic plasticity function, an effect that could potentially extend the window for gene therapy treatment to multiple developmental stages.

Cortico-Striatal Co-Culture was performed. In brief, primary cortico-striatal co-cultures were prepared as previously described in the art. Striatal tissues were dissected from P0 Shank3^{InsG3680/InsG3680} mutants. Cortical tissues were dissected from P0 wild-type pups. Tissue was digested with papain (Worthington Biochemical Corporation) and dissociated with a small glass Pasteur pipette. Approximately 5 million striatal medium spiny neurons (MSN) were electroporated with the Mouse Neuron Nucleofector™ Kit (Lonza) with either 1 µg of AAV-hSyn-GFP plasmid alone or 1 µg of AAV-hSyn1-GFP plasmid mixed with 2 µg of AAV-hSynl-minishank3 plasmid. The striatal MSNs and cortical neurons were then mixed at a ratio of 3:1 and plated onto 12 mm coverslips pre-coated with Poly D-lysine/Laminin (Neuvitro, GG-12-1.5-laminin) at a density of 1×10⁵ cells/cm² inside a 24-well plate with Neurobasal A medium (Invitrogen) supplemented with 0.5 mM glutamine (Invitrogen), 1X B27(Invitrogen), 50 µg/mL penicillin/streptomycin (Invitrogen), 50 ng/mL BDNF (R&D Systems), and 30 ng/mL GDNF (R&D Systems). After initial plating, half of the medium was exchanged with fresh medium without BDNF and GDNF every 3-4 days.

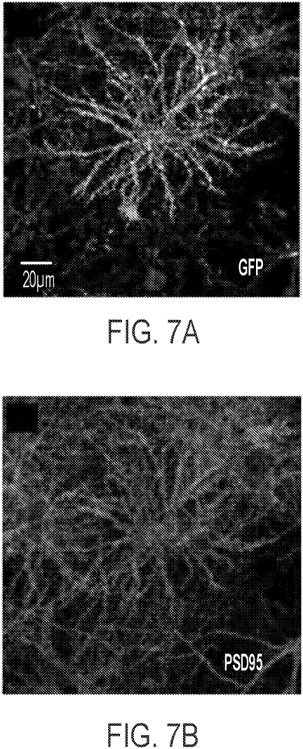

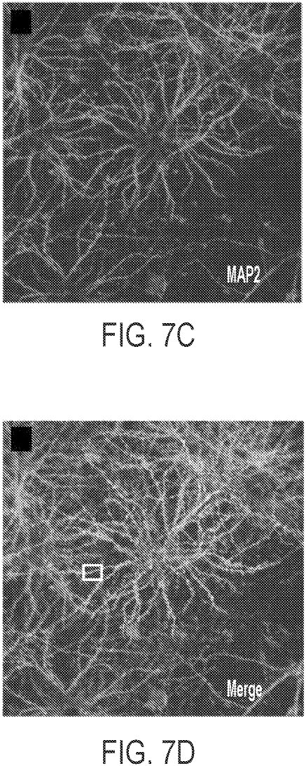

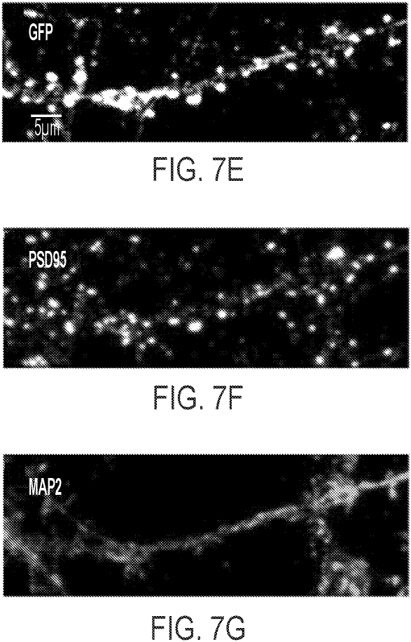

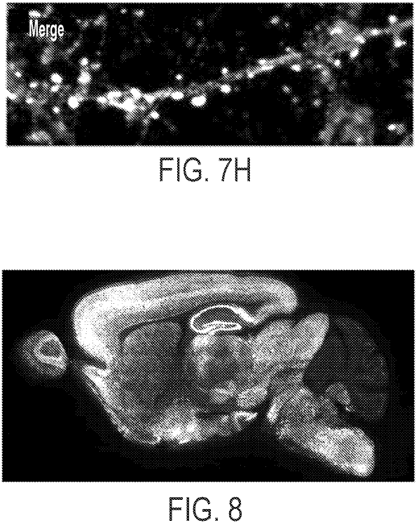

To examine whether the miniShank3 maintained key functions of Shank3, its expression and localization in cultured neurons from Shank3 mutant mice was first tested. Since Shank3 is a synaptic protein, it was critical that the miniShank3 was localized to synapses, similar to the endogenous Shank3 protein. It was found that when GFP-tagged miniShank3v1 was expressed in neurons of cortico-striatal co-culture, GFP-miniShank3 was precisely localized to synapses, indicated by its colocalization with postsynaptic marker PSD95 (FIGS. 7A-7H). Thus, miniShank3 retained this key feature of a synaptic protein.

### Example 3: Functional Expression of miniShank3 in Mouse Models

To examine whether the miniShank3 captures the full function of the endogenous Shank3, the ability of miniShank3-v1 in restoring neuronal function in Shank3 mutant mice was tested. It was previously demonstrated that Shank3 mutant mice exhibit deficits in the molecular composition of the postsynaptic density (PSD), including abnormalities in the electrophysiological properties of synapses and behavior. The ability of miniShank3-v1 in restoring each of these defects in Shank3 InsG3680 mutant mice was tested. This mouse mimics the InsG3680 mutation found in human ASD patients. Previous studies have shown that these mice exhibited molecular, electrophysiological and behavioral defects relevant to ASD. Thus, this model was used to test whether miniShank3-v1 could restore the defects observed in these mutant mice.

GFP-miniShank3-v1 was cloned into the pHP.eB AAV vector. AAV-GFP-miniShank3-v1 viruses were prepared and administered to postnatal day 0 to 2 (P0-P2) mice through facial vein injection. It was found that at the dose of 6.42E+11 viral genomes (vg) per mouse, GFP-miniShank3-v1 was highly expressed in the vast majority of neurons in the brain (FIG. 8). AAV-hSyn1-GFP was injected as control.

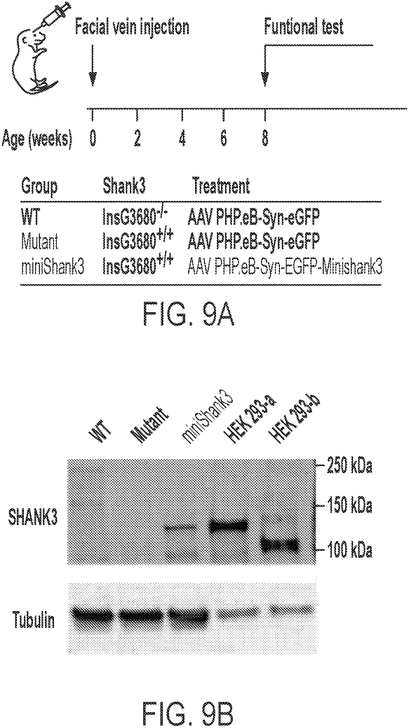

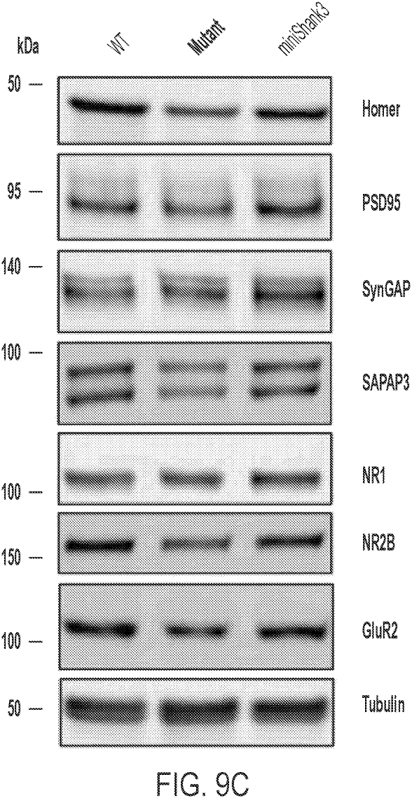

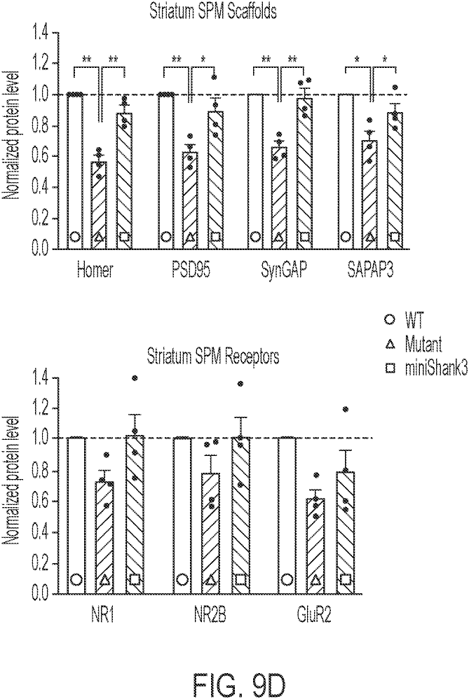

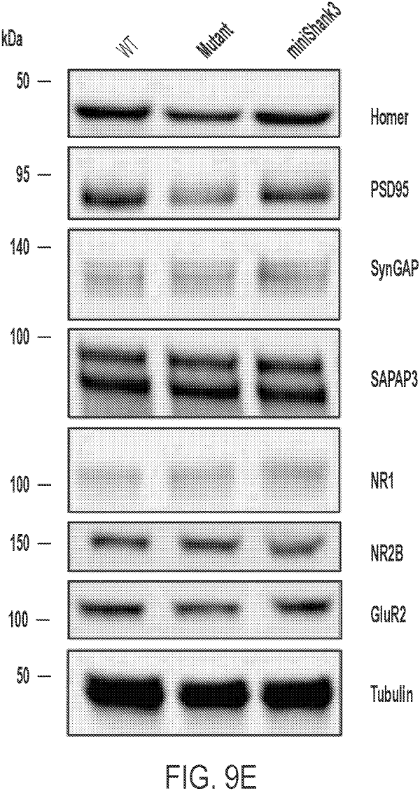

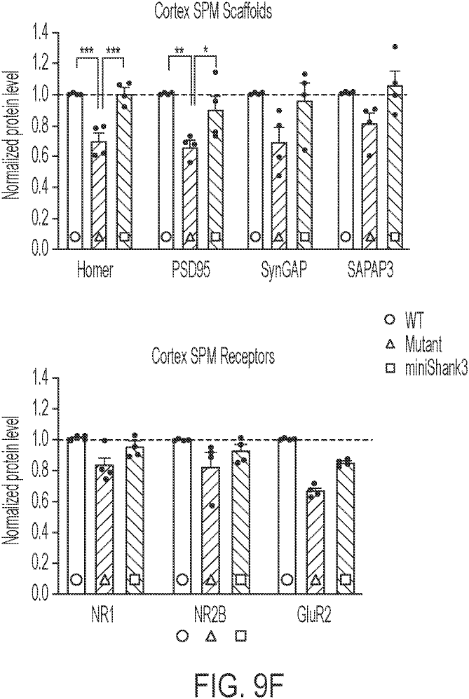

Using standard biochemical approaches, PSD was isolated from brains of wildtype mice injected with AAV-hSyn1-GFP, Shank3 mutant mice injected with AAV-hSyn1-GFP, and Shank3 mutant mice injected with AAV-GFP-miniShank3-v1 (FIG. 9A). Western blot assays were used to detect the levels of various synaptic proteins in the PSD (FIG. 9B). As previously reported, compared to wildtype mice, levels of several synaptic proteins, including Homer, PSD95, SynGap1, SAPAP3, NR1, NR2B, and GluR2, were reduced in the PSD of Shank3 mutant mice (FIGS. 9C-9F). The expression of miniShank3-v1 restored the expression levels of these synaptic proteins to the wildtype level, indicating that miniShank3-v1 was fully functional in restoring the molecular composition of the PSD (FIGS. 9C-9F). In conclusion, miniShank3 restores molecular defects in postsynaptic density (PSD) in Shank3 mutant mice.

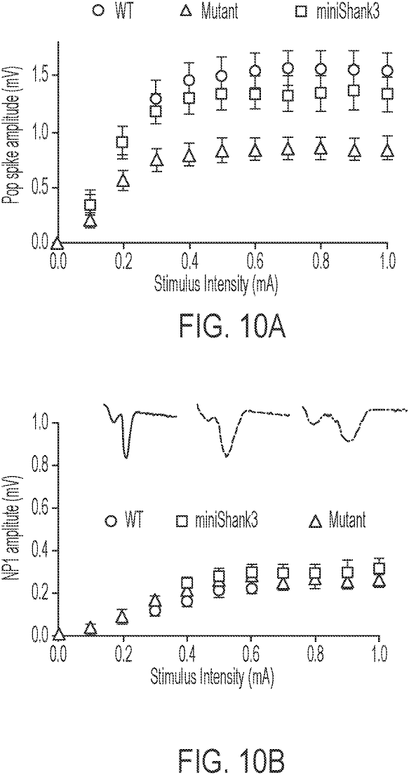

Shank3 is highly expressed in the striatum. Cortico-striatal synaptic communication is defective in Shank3 mutant mice. To test whether miniShank3-v1 could restore synaptic defects, electrophysiological recordings of cortico-striatal synaptic circuitry in acute brain slices from Shank3 mutant mice were performed. As previously reported, field population spikes were significantly reduced in Shank3 mutant mice when compared with controls (FIG. 10A). This defect in Shank3 mutant mice was rescued by miniShank3-v1 expression (FIG. 10A). Presynaptic function was not altered, as indicated by the relationship of stimulation intensity to the amplitude of the action potential component of the response termed negative peak 1 (NP1; FIG. 10B). These results indicated that the reduction in total field responses was most likely due to a postsynaptic impairment in synaptic function and/or a reduction in the number of functional synapses, and these defects could be effectively corrected by AAV-mediated miniShank3-v1 expression at P0. In conclusion, miniShank3 restores cortico-striatal synaptic defects in Shank3 mutant mice.

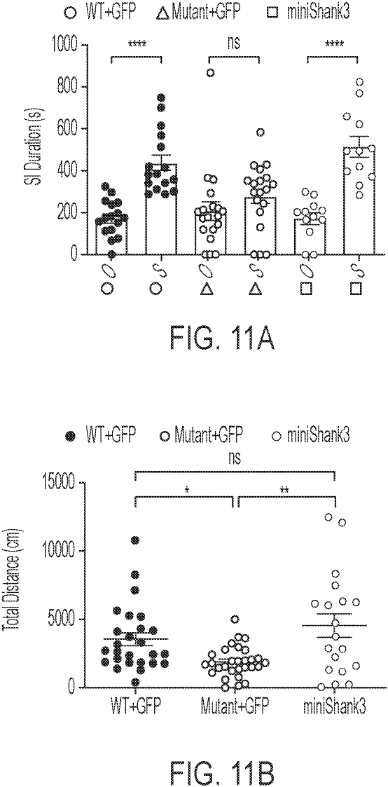

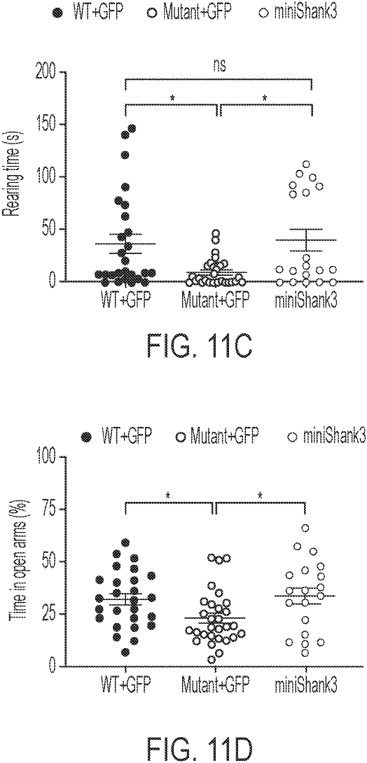

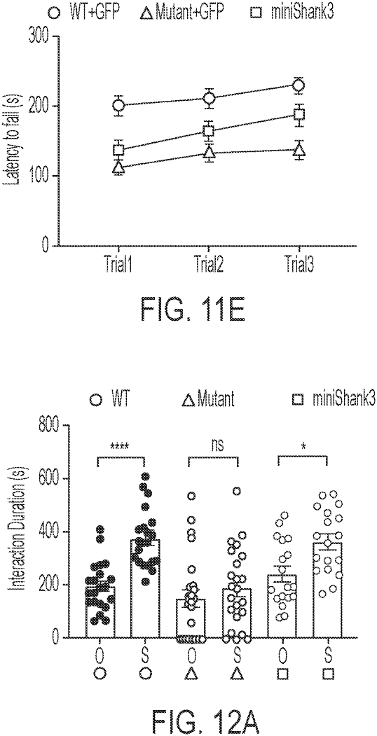

Previous studies have shown that Shank3 mutant mice exhibit several behavioral phenotypes relevant to symptoms seen in patients with Phelan-McDermid syndrome or Shank3 mutations. It was found that miniShank3-v1 treatment at P0 fully rescued all the tested behavioral deficits in Shank3 mutant mice except performance on a single assay of motor learning that showed only a trend of improvement. Treatment with miniShank3-v1 fully rescued social interaction deficits measured by 3-chamber social interaction assay (FIG. 11A), motor activity deficits measured by travel distance in open field test (FIG. 11B), explorative behavior deficits measured by rearing time in open field test (FIG. 11C), and anxiety-like behavior showing in elevated zero maze (FIG. 11D). However, motor learning deficit in mutant Shank3 was only slightly improved in rotarod test (FIG. 11E). This was likely related to very low levels of expression of miniShank3 in the cerebellum when injected at P0 because cerebellum development in mice starts postnatally which limited the AAV infection at P0. In conclusion, miniShank3 restores behavioral defects in Shank3 mutant mice.

### Example 4: Determination of the Therapeutic Timing for miniShank3 Gene Therapy

Previous studies demonstrated that there is a critical developmental time window for rescuing certain behaviors in Shank3 mutants. To determine the effective therapeutic window for AAV-mediated delivery of miniShank3, intravenous viral injection at different developmental stage was conducted and the effects on behavior during adulthood were assayed. All behavioral experiments were performed at least 8 weeks after AAV injection to the mice. In brief, wild type mice, Shank3 mutant mice, and Shank3 mutant mice with Shank3 treatment at different ages (P0, P2, P7, and P28) were assigned to experimental groups. Mice at P0 and P2 were intravenously injected with 20 µl of injection mix consisting of 6.0 ×10¹¹ total virus genome (vg) of AAV-PHP.eB-hSyn-GFP or AAV-PHP.eB-hSyn-GFP-MiniShank3 diluted in sterile saline. For mice at P0-P2 of age, facial vein injection was used. Mice at P7 and P28 were intravenously injected with 7.0×10¹¹ total virus genome (vg) of AAV-PHP.eB-hSyn-GFP or AAV-PHP.eB-hSyn-GFP-MiniShank3. For mice at P7 and P28 of age, retroorbital injection or intracerebroventricular (ICV) injection was used.

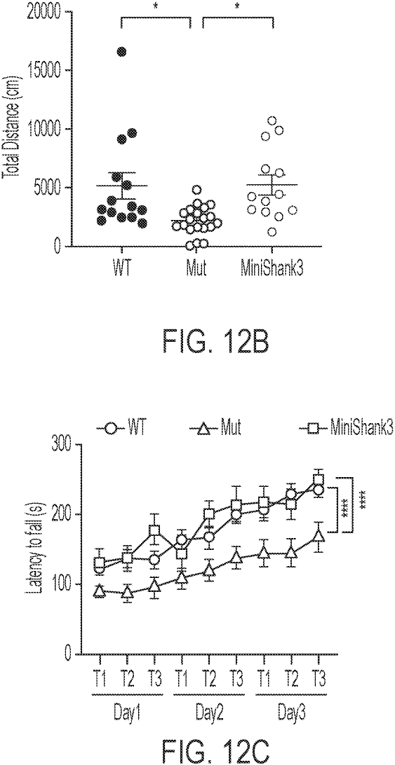

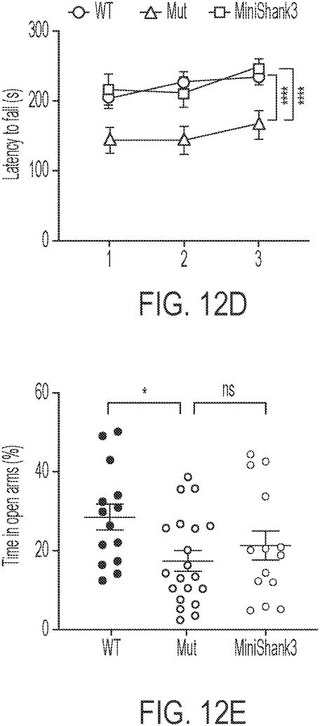

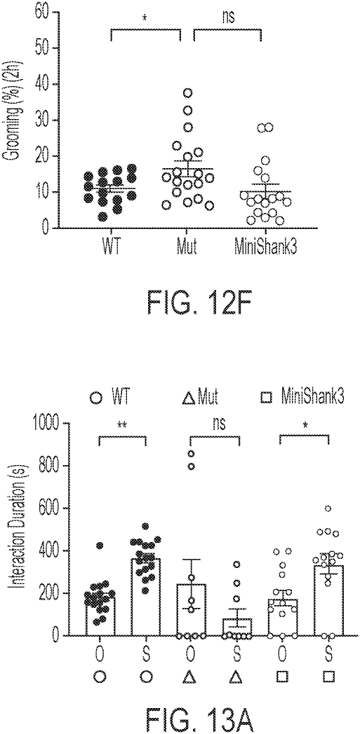

MiniShank3 treatment at postnatal day (P28) fully rescued social behavior impairments in Shank3 InsG3680 mutants, with treated mice showing a strong preference for the stranger mice, unlike untreated mutants that showed no preference (FIG. 12A). The P28-treated miniShank3 mice also showed significantly increased locomotor activity (FIG. 12B) compared to mutant mice. Treatment with miniShank3 at P28 was found to significantly improve motor learning (FIG. 12C) and motor coordination (FIG. 12D). In contrast to social and motor behaviors, miniShank3 treatment at P28 showed a minimal effect on anxiety-like behavior and repetitive grooming (FIG. 12D). In the elevated zero maze, miniShank3-treated mice showed no significant difference from the mutant mice in exploration time in the open arms (FIG. 12F) and in the grooming assay the miniShank3-treated mice showed only a slight reduction in grooming behavior (FIG. 12F).

Previous studies describing the genetic rescue of Shank3 expression in adult mice showed that grooming phenotypes were reversible in adults and cortico-striatal-thalamo-cortical circuitry dysfunction were strongly implicated in repetitive/compulsive-like behaviors. To address whether the lack of rescue of these behavioral phenotypes in mutants treated with miniShank3 at P28 was due to poor miniShank3 expression at grooming-related brain regions, the biodistribution of miniShank3 was examined by assessing GFP expression in the P28-treated animals and it was found that GFP expression was extremely low in thalamus and limited in the striatum. Thus, while miniShank3 treatment at P28 selectively rescued deficits in social behavior, locomotion, and motor coordination, improved strategies for AAV-based targeting of thalamus and striatum at this age could enable additional therapeutic benefits for anxiety and repetitive behaviors.

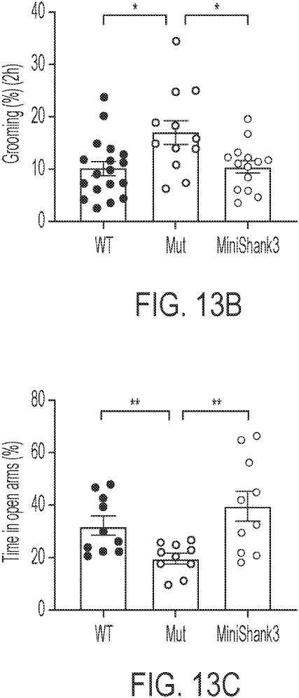

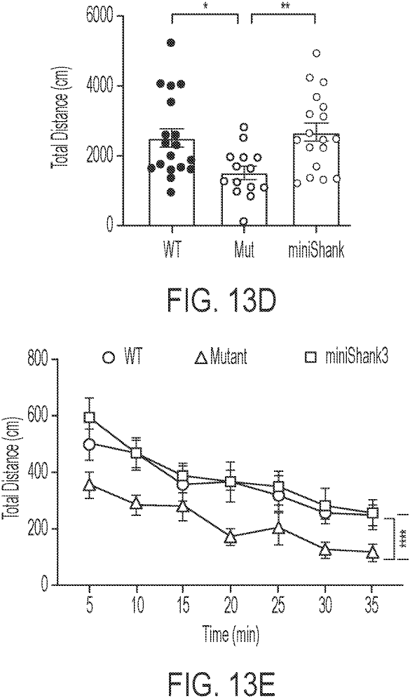

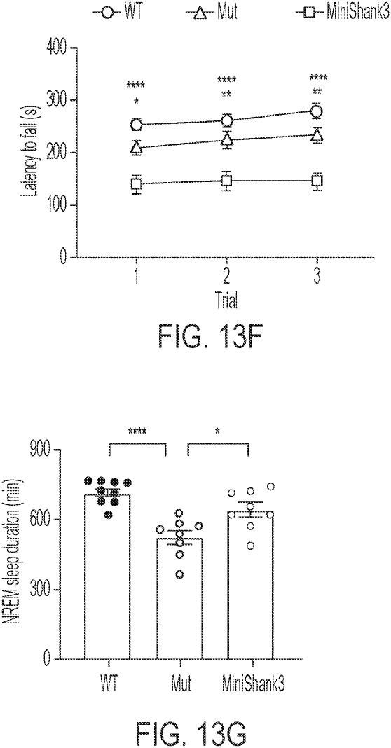

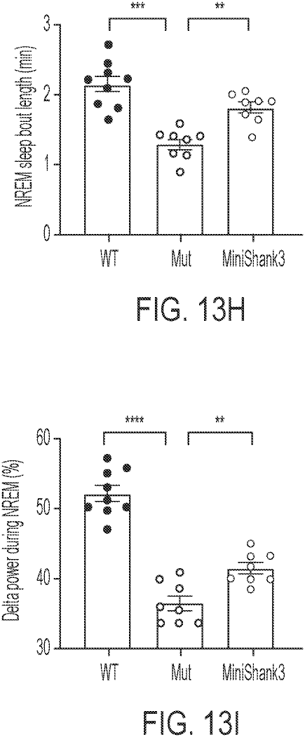

Mutant mice dosed with miniShank3 at postnatal day 7 (P7) showed a strengthened preference for stranger mice compared to their mutant littermates, similar to what was observed with P0-P2 injection (FIG. 13A). Electrophysiological studies showed that miniShank3 delivery at P7 was sufficient to restore the cortical-striatal synaptic defects observed in Shank3mutant mice. Cortical-striatal circuit dysfunction was strongly implicated in the repetitive and compulsive behaviors associated with autism and obsessive-compulsive disorder and Shank3 mutants displayed overgrooming phenotypes. To test this theory, individual WT mice, Shank3 mutant mice, and Shank3 mutant mice treated with miniShank3 at P7 were monitored for 2 hours and the percentage of time spent grooming during the session was quantified. It was found that Shank3 mutant mice spent a significantly increased percentage of time grooming compared to WT mice. However, miniShank3-treated mice spent a significantly reduced amount of time grooming and were similar to WT animals (FIG. 13B).

MiniShank3 treatment at P7 fully rescued the locomotor phenotypes observed in Shank3 mutants, as measured by the total distance traveled in the open-field test (FIG. 13C), anxiety-like behavior in the elevated zero maze (FIGS. 13D-13E). In contrast to P0-P2 (postnatal day 0 - postnatal day 2) delivery of miniShank3, Shank3mutant mice treated miniShank3 at P7 performed significantly better in both motor learning and motor coordination than the mutant littermates (FIG. 13F), suggesting the motor deficits observed in Shank3 mutants were reversible by AAV- mediated miniShank3 gene therapy if given at the appropriate developmental stage. Together, these behavioral results show that miniShank3 gene therapy at P7 was able to effectively rescue all reported behavioral phenotypes in Shank3 InsG3680 mutant animals.

### Example 5: Systemic Delivery of miniShank3 at P7 Results in Improvement of Sleep Disturbance in Shank3 InsG3680 Mutants

It has been shown that patients with Phelan-McDermid syndrome and other individuals with SHANK3 mutations often exhibit severe sleep disturbances including have trouble falling and staying asleep. For example, SHANK3 mutation in macaques exhibit notable sleep disruption. To assess sleep disturbance in the Shank3 InsG3680 mutant mice, EEG/EMG related surgery was performed and the signals from all three experimental groups were examined to determine whether sleep was affected by mutation of Shank3. InsG3680 homozygotes displayed reduced NREM sleep duration (FIG. 13G) with shorter bout length (FIG. 13H) than WT controls, and an attenuated power of delta rhythm (1-4 Hz) in the frontal EEG during NREM sleep (FIG. 13I). These results showed severe sleep disturbances in Shank3 mutant mice. MiniShank3 injected at P7 significantly mitigated both the reduction in NREM sleep (FIG. 13G) and the decreased sleep bout lengths observed in untreated mutants (FIG. 13H). In addition, miniShank3 injected at P7 partially rescued attenuated delta power (FIG. 13I). Together, these results showed that miniShank3 can attenuate sleep disruptions in homozygous Shank3 InsG3680 mice and are consistent with the broad ability of miniShank3 to rescue ASD-related behavioral phenotypes.

### Example 6: MiniShank3 Treatment at P7 Does Not Cause Seizure Activity in Shank3 InsG3680 Mutants

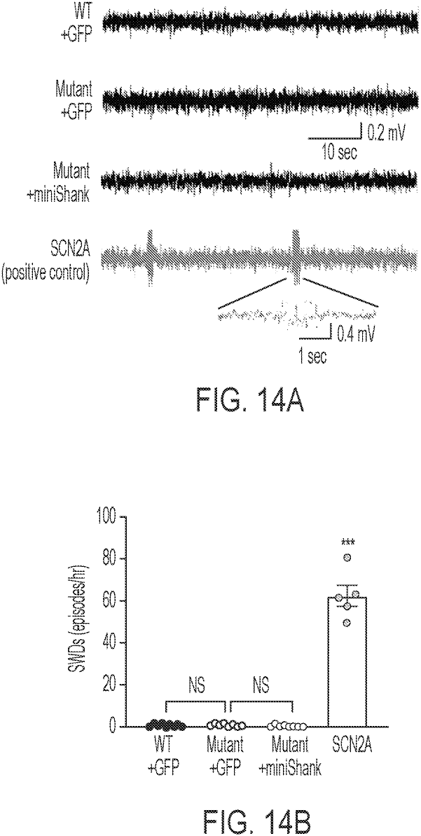

To test potential effects of the miniShank3 treatment on seizure activity, WT mice, Shank3 mutants injected with control virus, Shank3 mutants injected with miniShank3, and Scn2a mutants were included in the study. For EEG analysis, indicators such as sleep monitoring, spontaneous seizures, and audiogenic seizures were performed. No spontaneous epileptic EEG abnormalities were observed in Shank3 InsG3680 mutant mice as evidenced by stable EEG baseline during 24 hours of recording (representative traces, FIG. 14A). The susceptibility of Shank3 mutants to induce audiogenic seizures (124 dB acoustic stimuli) was examined and it was found that no behavioral signs of audiogenic seizures in mutant mice were present. To assess the safety of miniShank3, spontaneous epileptic EEG abnormalities were investigated in mutant mice injected with miniShank3 at P7 and no detectable hyperexcitatory activities on EEG were observed (FIG. 14A). In addition, Shank3 InsG3680 mutant mice and mutant mice injected miniShank3 did not show spike-and-wave discharges (SWDs), an EEG signature of absence seizures. However, analysis of animals with a heterozygous mutation in Scn2a, which resulted in frequent absence seizures, found about 60 episodes of SWD per hour, validating the effectiveness of the analysis pipeline (FIG. 14B). Together, these results show that Shank3 InsG3680 mutants did not show epileptiform activity and that miniShank3 expression did not result in detectable seizures.

### Example 7: Treatment of Adult Human Subjects With miniShank3

Adult human subjects who have, are suspected of having, or at risk of having a neurodevelopment disorder, such as an autism spectrum disorder (ASD), or Phelan-McDermid syndrome can be treated by a miniShank3 using the methods, vectors, and non-naturally occurring polynucleotides as described herein. The adult human subjects may include any male or female adults who are 16 years of age or older. An adult who is younger than 25 years of age may be preferred for the miniShank3 treatment as disclosed herein. The administration and delivery methods may include facial vein injection and intracerebroventricular injection as disclosed herein. Other methods that are known by a skilled person in the art can also be used. An adult human subject is treated with miniShank3 delivered by a viral vector, such as AAV. For example, the adult human subject can be treated with an AAV vector comprising the sequence of SEQ ID NO: 21 expressing a miniShank3 transgene. Without wishing to be bound by any theory, the dose for treatment can be between 1×10¹⁰and 1×10¹² viral genomes (vg). A skilled person in the art would understand that various factors such as gender, weight, age, the status of the disease, and the type of the disease can be considered when determining a dose for a specific adult. The dose can be outside the range of 1×10¹⁰and 1×10¹² vg. The route of administration may be, for example, intravenous, facial intravenous, intracranial, intracerebroventricular, intraocular, or intrathecal.

### Example 8: Treatment of Non-adult Human Subjects With miniShank3

Non-adult human subjects who have, are suspected of having, or at risk of having a neurodevelopment disorder, such as an autism spectrum disorder (ASD), or Phelan-McDermid syndrome can be treated by a miniShank3 using the methods, vectors, and non-naturally occurring polynucleotides as described herein. The non-adult human subjects may include any males or females who are under 16 years of age or younger. Without wishing to be bound by any theory, a human subject who is 10 years of age or younger such infants or toddlers may be preferred for the miniShank3 treatment as disclosed herein. The administration and delivery methods may include facial vein injection and intracerebroventricular injection are disclosed herein. Other methods that are known by a skilled person in the art can also be used. A non-adult human subject is treated with miniShank3 delivered by a viral vector, such as AAV. For example, the non-adult human subject can be treated with an AAV vector comprising the sequence of SEQ ID NO: 21 expressing a miniShank3 transgene. Without wishing to be bound by any theory, the dose for treatment can be between 1×10¹⁰and 1×10¹² viral genomes (vg). A skilled person in the art would understand that various factors such as gender, weight, age, the status of the disease, and the type of the disease can be considered when determining a dose for a specific adult. The dose can be outside the range of 1×10¹⁰and 1×10¹² vg. The route of administration may be, for example, intravenous, facial intravenous, intracranial, intracerebroventricular, intraocular or intrathecal. The route of administration may be in utero if the non-adult human subject is at the fetal or prenatal stage of development.

### Example 9: Treatment of Human Subjects With miniShank1 or miniShank2

Human subjects who have, are suspected of having, or at risk of having a neurodevelopment disorder, an autism spectrum disorder (ASD), or Phelan-McDermid syndrome can be treated by a miniShank1 or miniShank2 using the methods, vectors, and non-naturally occurring polynucleotides as described herein. The adult human subjects may include any male or female human subjects. The method of treatment will be similar to the methods described herein, with the exception that the miniShank3 construct (SEQ ID NOs: 1-4 and 21) are modified to comprise Shank1 (miniShank1) or Shank2 (miniShank2). An adult or non-adult human subject is treated with miniShank1 or miniShank2 delivered by a viral vector, such as AAV. For example, the adult human or non-adult human subject can be treated with an AAV vector comprising the sequence of SEQ ID NO: 21 expressing a miniShank3 transgene. Without wishing to be bound by any theory, the dose for treatment can be between 1×10¹⁰and 1×10¹² viral genomes (vg). A skilled person in the art would understand that various factors such as gender, weight, age, the status of the disease, and the type of the disease can be considered when determining a dose for a specific adult. The dose can be outside the range of 1×10¹⁰and 1×10¹² vg. The route of administration may be, for example, intravenous, facial intravenous, intracranial, intracerebroventricular, intraocular, or intrathecal. The route of administration may be in utero if the non-adult human subject is at the fetal or prenatal stage of development.

### Example 10: Delivery of miniShank3 Using a Lentivirus Viral Vector

Human subjects who have, are suspected of having, or at risk of having a neurodevelopment disorder, such as an autism spectrum disorder (ASD), or Phelan-McDermid syndrome can be treated by a miniShank1 or miniShank2 using the methods, vectors, and non-naturally occurring polynucleotides as described herein. An adult or non-adult human subject is treated with miniShank3 delivered by a viral vector, such as a lentivirus. Without wishing to be bound by any theory, the dose for treatment can be between 1×10¹⁰and 1×10¹² viral genomes (vg). A skilled person in the art would understand that various factors such as gender, weight, age, the status of the disease, and the type of the disease can be considered when determining a dose for a specific adult. The dose can be outside the range of 1×10¹⁰and 1×10¹² vg. The route of administration may be, for example, intravenous, facial intravenous, intracranial, intracerebroventricular, intraocular, or intrathecal. The route of administration may be in utero if the non-adult human subject is at the fetal or prenatal stage of development.

TABLE 1

Mouse and Human miniShank3 Sequences and Vector Sequences

| SEQ ID NO: | Description | Sequence |
|---|---|---|
| 1 | Mouse miniShank3-V1 | atgggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggag |
| | | cgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga |
| 2 | Human miniShank3-V1 | atgggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga |
| 3 | Mouse miniShank3-V2 | atggacggccccggggccagcgccgtggtcgtgcgcgtcggcatcccggacctgcaacaaacgaagtgcctgcgtctggacccaaccgcgcccgtgtgggccgccaagcagcgtgtgctctgcgccctcaatcatagccttcaagacgcgctcaactacgggctattccagcctccctcccggggtcgcgccggcaagttcctggatgaagagcggctcttacaggactacccgcctaacctggacacgcccctgccctatctggagttccgatacaagcggagagtttatgcccagaacctcatagatgacaagcagtttgcaaagctacacacaaaggcaaacctgaagaagttcatggactatgtccagctacacagcacagataaggtggcccgcctgctggacaaggggctggaccccaatttccatgaccctgactcaggagagtgccctctgagccttgcggcacagttggacaacgccactgacctcctgaaggttctccgcaacggcggtgctcatctggacttccggacccgagatgggctgacagccgtccactgtgctacccgccagcggaacgcaggggcattgacgaccctgctggacctgggggcttcgcctgactacaaggacagccgcggcctgacgcccctgtaccatagtgccctagggggcggggatgccctctgttgcgagctgcttctccatgatcatgcacagctggggaccactgatgagaatggttggcaagagatccatcaggcctgtcgctttggacacgtgcagcacctggagcaccttttgttctatggggccaacatgggtgctcagaatgcctcggg |
| | | aaacacagccctgcacatctgtgccctctacaaccaggagagttgcgcgcgcgtcctgcttttccgtggtgccaacaaggacgtccgcaattacaacagccagacagccttccaggtggccattattgcagggaactttgagcttgccgaggtaatcaagacccacaaagactccgatgtcgtaccattcagggaaacccccagctatgcaaagcgacggcgtctggctggcccgagtggcctggcatccccacggcccttacagcgctcagccagtgatatcaacctgaaaggtgcgccgccgccccggggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga |
| 4 | Human miniShank3-V2 | atggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatg |
| | | cgaagcggcggcgactggctggccccagtggcttggcatcccctcggcctctgcagcgctcagccagcgatatcaacctgaagggggcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga |
| 7 | AAV- hSyn-EGFP-miniShank3 (5′ITR-hSyn-EGFP-miniShank3-polyA-WPRE-3′ITR) | cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcagcgctgttaacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggc |
| | | atcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggaggaattcacggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctgagcggccatcgtcgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggcca |
| | | ctccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt |
| 8 | AAV- hSyn-GFP (5′ITR-hSyn-EGFP-polyA-WPRE-3′ITR) | cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcagcgctgttaacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggc |
| | | gtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgattt |
| | | aaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtccgtcgggtttcgccacctctgacttgagcgtcgatttttgtgatgctcgtcaggggggcggagcctatggaaaaacgccagcaacgcggcctttttacggttcctggccttttgctggccttttgctcacatgt |
| 9 | AAV- hSyn-nCre-EGFP (5′ITR-hSyn-EGFP-Cre-polyA-WPRE-3′ITR) | tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcaccatgcctaagaagaagagaaaggtatgcaggtcacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatgttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggg |
| | | actttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg |
| 10 | AAV-PHP.eBhSyn-Delta-nCre-EGFP (5′ITR-hSyn-EGFP-Delta-Cre-polyA-WPRE-3′ITR) | cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcaccatgcctaagaagaagagaaaggtatgcaggtcacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgagttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaac |
| | | aaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt |
| 11 | Protein sequence encoded by the transgene of SEQ ID NO: 7 (AAV-hSyn-EGFP-miniShank3) | MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGGIHGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS* |
| 12 | Protein sequence encoded by the transgene of SEQ ID NO: 8 (AAV-hSyn-GFP) | MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK* |
| 13 | Protein sequence encoded by the transgene of SEQ ID NO: 9 (AAV-hSyn-nCre-EGFP) | MPKKKRKVCRSHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDVNVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK* |
| 14 | Protein sequence encoded by the transgene of SEQ ID NO: 10 (AAV-PHP.eBhSyn-Delta-nCre-EGFP) | MPKKKRKVCRSHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRVNVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK* |
| 17 | Mouse miniShank3-V1 protein sequence encoded by SEQ ID NO: 1 | MGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS* |
| 18 | Human miniShank3-V1 protein | MGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEF |
| | sequence encoded by SEQ ID NO: 2 | TPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS* |
| 19 | Mouse miniShank3-V2 protein sequence encoded by SEQ ID NO: 3 | MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS* |
| 20 | Human miniShank3-V2 protein sequence encoded by SEQ ID NO: 4 | MDGPGASAVVVRVGIPDLQQTKCLRLDPAAPVWAAKQRVLCALNHSLQDALNYGLFQPPSRGRAGKFLDEERLLQEYPPNLDTPLPYLEFRYKRRVYAQNLIDDKQFAKLHTKANLKKFMDYVQLHSTDKVARLLDKGLDPNFHDPDSGECPLSLAAQLDNATDLLKVLKNGGAHLDFRTRDGLTAVHCATRQRNAAALTTLLDLGASPDYKDSRGLTPLYHSALGGGDALCCELLLHDHAQLGITDENGWQEIHQACRFGHVQHLEHLLFYGADMGAQNASGNTALHICALYNQESCARVLLFRGANRDVRNYNSQTAFQVAIIAGNFELAEVIKTHKDSDVVPFRETPSYAKRRRLAGPSGLASPRPLQRSASDINLKGAPPPRGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTR |
| | | KPEEDGARRRAPPPPKRAPSTTLTLRSKSMTAELEELASIRSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS* |
| 21 | AAV-hSyn1-HumanMiniS hank3-V1 (5′ITR-hSyn-WPRE-hgH polyA-3′ITR) | cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcatgGGCCCGAAGCGGAAACTTTACAGCGCCGTCCCCGGCCGCAAGTTCATCGCCGTGAAGGCGCACAGCCCGCAGGGTGAAGGCGAGATCCCGCTGCACCGCGGCGAGGCCGTGAAGGTGCTCAGCATTGGGGAGGGCGGTTTCTGGGAGGGAACCGTGAAAGGCCGCACGGGCTGGTTCCCGGCCGACTGCGTGGAGGAAGTGCAGATGAGGCAGCATGACACACGGCCTGAAACGCGGGAGGACCGGACGAAGCGGCTCTTTCGGCACTACACAGTGGGCTCCTACGACAGCCTCACCTCACACAGCGATTATGTCATTGATGACAAAGTGGCTGTCCTGCAGAAACGGGACCACGAGGGCTTTGGTTTTGTGCTCCGGGGAGCCAAAGCAGAGACCCCCATCGAGGAGTTCACGCCCACGCCAGCCTTCCCGGCGCTGCAGTATCTCGAGTCGGTGGACGTGGAGGGTGTGGCCTGGAGGGCCGGGCTGCGCACGGGAGACTTCCTCATCGAGGTGAACGGGGTGAACGTGGTGAAGGTCGGACACAAGCAGGTGGTGGCTCTGATTCGCCAGGGTGGCAACCGCCTCGTCATGAAGGTTGTGTCTGTGACAAGGAAGCCAGAAGAGGACGGGGCTCGGCGCAGAGCCCCACCGCCCCCCAAGAGGGCCCCCAGCACCACACTGACCCTGCGCTCCAAGTCCATGACAGCTGAGCTCGAGGAACTTGCCTCCATTCGGAGCTCAGAGGAAGAGCCAGAGCTGGTGTTTGCTGTGAACCTGCCACCTGCCCAGCTGTCGTCCAGCGATGAGGAGACCAGGGAGGAGCTGGCCCGAATTGGGTTGGTGCCACCCCCTGAAGAGTTTGCCAACGGGGTCCTGCTGGCCACCCCACTCGCTGGCCCGGGCCCCTCGCCCACCACGGTGCCCAGCCCGGCCTCAGGGAAGCCCAGCAGTGAGCCACCCCCTGCCCCTGAGTCTGCAGCCGACTCTGGGGTGGAGGAGGCTGACACACGCAGCTCCAGCGACCCCCACCTGGAGACCACAAGCACCATCTCCACGGTGTCCAGCATGTCCACCTTGAGCTCGGAGAGCGGGGAACTCACTGACACCCACACCTCCTTCGCTGACGGACACACTTTTCTACTCGAGAAGCCACCAGTGCCTCCCAAGCCCAAGCTCAAGTCCCCGCTGGGGAAGGGGCCGGTGACCTTCAGGGACCCGCTGCTGAAGCAGTCCTCGGACAGCGAGCTCATGGCCCAGCAGCACCACGCCGCCTCTGCC |
| | | GGGCTGGCCTCTGCCGCCGGGCCTGCCCGCCCTCGCTACCTCTTCCAGAGAAGGTCCAAGCTATGGGGGGACCCCGTGGAGAGCCGGGGGCTCCCTGGGCCTGAAGACGACAAACCAACTGTGATCAGTGAGCTCAGCTCCCGCCTGCAGCAGCTGAACAAGGACACGCGTTCCCTGGGGGAGGAACCAGTTGGTGGCCTGGGCAGCCTGCTGGACCCTGCCAAGAAGTCGCCCATCGCAGCAGCTCGGCTCTTCAGCAGCCTCGGTGAGCTGAGCTCCATTTCAGCGCAGCGCAGCCCCGGGGGCCCGGGCGGCGGGGCCTCGTACTCGGTGAGGCCCAGTGGCCGCTACCCCGTGGCGAGACGCGCCCCGAGCCCGGTGAAGCCCGCGTCGCTGGAGCGGGTGGAGGGGCTGGGGGCGGGCGCGGGGGGCGCAGGGCGGCCCTTCGGCCTCACGCCCCCCACCATCCTCAAGTCGTCCAGCCTCTCCATCCCGCACGAGCCCAAGGAGGTGCGCTTCGTGGTGCGCAGCGTGAGCGCGCGCAGTCGCTCCCCCTCGCCGTCGCCGCTGCCCTCGCCCGCGTCCGGCCCCGGCCCCGGCGCCCCCGGCCCACGCCGACCCTTCCAGCAGAAGCCGCTGCAGCTCTGGAGCAAGTTCGACGTGGGCGACTGGCTGGAGAGCATCCACCTAGGCGAGCACCGCGACCGCTTCGAGGACCATGAGATAGAAGGCGCGCACCTACCCGCGCTTACCAAGGACGACTTCGTGGAGCTGGGCGTCACGCGCGTGGGCCACCGCATGAACATCGAGCGCGCGCTCAGGCAGCTGGACGGCAGCTGAcggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctca |
| | | cccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt |
| 22 | hSyn1 promoter | gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctaga |

### REFERENCES

1. Prasad C, Prasad AN, Chodirker BN, Lee C, Dawson AK, Jocelyn LJ, Chudley AE. (2000) Genetic evaluation of pervasive developmental disorders: the terminal 22q13 deletion syndrome may represent a recognizable phenotype. Clin Genet 57:103-109.

2. Precht KS, Lese CM, Spiro RP, Huttenlocher PR, Johnston KM, Baker JC, Christian SL, Kittikamron K, Ledbetter DH. (1998) Two 22q telomere deletions serendipitously detected by FISH. J Med Genet 35:939-942.

3. Manning MA, Cassidy SB, Clericuzio C, Cherry AM, Schwartz S, Hudgins L, Enns GM, Hoyme HE. (2004) Terminal 22q deletion syndrome: a newly recognized cause of speech and language disability in the autism spectrum. Pediatrics 114, 451-457.

4. Wilson HL, Wong AC, Shaw SR, Tse WY, Stapleton GA, Phelan MC, Hu S, Marshall J, McDermid HE. (2003) Molecular characterisation of the 22q13 deletion syndrome supports the role of haploinsufficiency of SHANK3/PROSAP2 in the major neurological symptoms. J Med Genet 40, 575-584.

5. Jeffries AR, Curran S, Elmslie F, Sharma A, Wenger S, Hummel M, Powell J (2005) Molecular and phenotypic characterization of ring chromosome 22. Am J Med Genet A 137:139-147.

6. Durand CM, Betancur C, Boeckers TM, Bockmann J, Chaste P, Fauchereau F, Nygren G, Rastam M, Gillberg IC, Anckarsäter H, Sponheim E, Goubran-Botros H, Delorme R, Chabane N, Mouren-Simeoni MC, de Mas P, Bieth E, Rogé B, Héron D, Burglen L, Gillberg C, Leboyer M, Bourgeron T. (2007) Mutations in the gene encoding the synaptic scaffolding protein SHANK3 are associated with autism spectrum disorders. Nat Genet 39, 25-27.

7. Moessner R, Marshall CR, Sutcliffe JS, Skaug J, Pinto D, Vincent J, Zwaigenbaum L, Fernandez B, Roberts W, Szatmari P, Scherer SW. (2007) Contribution of SHANK3 mutations to autism spectrum disorder. Am J Hum Genet 81, 1289-1297.

8. Gauthier J, Spiegelman D, Piton A, Lafrenière RG, Laurent S, St-Onge J, Lapointe L, Hamdan FF, Cossette P, Mottron L, Fombonne E, Joober R, Marineau C, Drapeau P, Rouleau GA. (2009) Novel de novo SHANK3 mutation in autistic patients. Am J Med Genet B Neuropsychiatr Genet 150B, 421-424.

9. Boccuto et al., Prevalence of SHANK3 variants in patients with different subtypes of autism spectrum disorders. Eur J Hum Genet. 2013 Mar;21(3):310-6.

10. Betancur C, Buxbaum JD. SHANK3 haploinsufficiency: a “common” but underdiagnosed highly penetrant monogenic cause of autism spectrum disorders. Mol Autism. 2013 Jun 11;4(1):17.

11. Du, Y., Weed, S.A., Xiong, W.C., Marshall, T.D. & Parsons, J.T. (1998) Identification of a novel cortactin SH3 domain-binding protein and its localization to growth cones of cultured neurons. Mol Cell Biol 18, 5838-5851 (1998).

12. Naisbitt S, Kim E, Tu JC, Xiao B, Sala C,Valtschanoff J, Weinberg RJ, Worley PF, Sheng M. (1999) Shank, a novel family of postsynaptic density proteins that binds to the NMDA receptor/PSD-95/GKAP complex and cortactin. Neuron 23: 569-82.

13. Boeckers TM, Winter C, Smalla KH, Kreutz MR, Bockmann J, Seidenbecher C, Garner CC, Gundelfinger ED. (1999a) Proline-rich synapse-associated proteins ProSAP1 and ProSAP2 interact with synaptic proteins of the SAPAP/GKAP family. Biochem Biophys Res Commun. 264:247-52.

14. Boeckers TM, Kreutz MR, Winter C, Zuschratter W, Smalla KH, Sanmarti-Vila L, Wex H, Langnaese K, Bockmann J, Garner CC, Gundelfinger ED. (1999b) Proline-rich synapse-associated protein-1/cortactin binding protein 1 (ProSAP1/CortBP1) is a PDZ-domain protein highly enriched in the postsynaptic density. J Neurosci. 19:6506-18.

15. Sheng, M. & Kim, E. (2000) The Shank family of scaffold proteins. J Cell Sci 113 ( Pt 11), 1851-1856.

16. Montgomery JM, Zamorano PL, Garner CC. (2004) MAGUKs in synapse assembly and function: an emerging view. Cell Mol Life Sci. 61:911-29

17. Kim, E. and M. Sheng (2004). PDZ domain proteins of synapses. Nat Rev Neurosci 5: 771-81.

18. McAllister AK. (2007) Dynamic aspects of CNS synapse formation. Annu Rev Neurosci. 30:425-50.

19. Tu JC, Xiao B, Naisbitt S, Yuan JP, Petralia RS, Brakeman P, Doan A, Aakalu VK, Lanahan AA, Sheng M, Worley PF. (1999) Coupling of mGluR/Homer and PSD-95 complexes by the Shank family of postsynaptic density proteins. Neuron 23, 583-592.

20. Valtschanoff, J.G. & Weinberg, R.J. (2011) Laminar organization of the NMDA receptor complex within the postsynaptic density. J Neurosci 21, 1211-1217.

21. Baron MK, Boeckers TM, Vaida B, Faham S, Gingery M, Sawaya MR, Salyer D, Gundelfinger ED, Bowie JU. (2006) An architectural framework that may lie at the core of the postsynaptic density. Science 311:531-5.

22. Kreienkamp, H.J. (2008) Scaffolding proteins at the postsynaptic density: shank as the architectural framework. Handb Exp Pharmacol, 365-380.

23. Hayashi MK, Tang C, Verpelli C, Narayanan R, Stearns MH, Xu RM, Li H, Sala C, Hayashi Y. (2009) The postsynaptic density proteins Homer and Shank form a polymeric network structure. Cell 137:159-71.

24. Roussignol G, Ango F, Romorini S, Tu JC, Sala C, Worley PF, Bockaert J, Fagni L. (2005) Shank expression is sufficient to induce functional dendritic spine synapses in aspiny neurons. J Neurosci 25, 3560-3570.

25. Sala C, Piëch V, Wilson NR, Passafaro M, Liu G, Sheng M. Regulation of dendritic spine morphology and synaptic function by Shank and Homer. (2001) Regulation of dendritic spine morphology and synaptic function by Shank and Homer. Neuron 31, 115-130.

26. Hung AY, Futai K, Sala C, Valtschanoff JG, Ryu J, Woodworth MA, Kidd FL, Sung CC, Miyakawa T, Bear MF, Weinberg RJ, Sheng M. (2008) Smaller dendritic spines, weaker synaptic transmission, but enhanced spatial learning in mice lacking Shank1. J Neurosci. 28:1697-708.

27. American Psychiatric Association. & American Psychiatric Association. Task Force on DSM-IV. Diagnostic and statistical manual of mental disorders : DSM-IV-TR, (American Psychiatric Association, Washington, DC, 2000.

28. Bertrand J, Mars A, Boyle C, Bove F, Yeargin-Allsopp M, Decoufle P. (2001) Prevalence of autism in a United States population: the Brick Township, New Jersey, investigation. Pediatrics 108:1155-1161.

29. Chakrabarti, S. & Fombonne, E. (2005) Pervasive developmental disorders in preschool children: confirmation of high prevalence. Am J Psychiatry 162:1133-1141.

30. Baird G, Simonoff E, Pickles A, Chandler S, Loucas T, Meldrum D, Charman T. (2006) Prevalence of disorders of the autism spectrum in a population cohort of children in South Thames: the Special Needs and Autism Project (SNAP). Lancet 368:210-215.

31. Rosenberg RE, Law JK, Yenokyan G, McGready J, Kaufmann WE, Law PA. (2009) Characteristics and concordance of autism spectrum disorders among 277 twin pairs. Arch Pediatr Adolesc Med 163:907-914.

32. Newschaffer CJ, Croen LA, Daniels J, Giarelli E, Grether JK, Levy SE, Mandell DS, Miller LA, Pinto-Martin J, Reaven J, Reynolds AM, Rice CE, Schendel D, Windham GC. (2007) The epidemiology of autism spectrum disorders. Annu Rev Public Health 28:235-258.

33. Veenstra-VanderWeele, J., Christian, S.L. & Cook, E.H. (2004) Autism as a paradigmatic complex genetic disorder. Annu Rev Genom Hum G. 5, 379-405.

34. Zoghbi, H.Y. [2003] Postnatal neurodevelopmental disorders: meeting at the synapse? Science 302:826-830.

35. Cline H. (2005) Synaptogenesis: a balancing act between excitation and inhibition. Curr Biol. 15:R203-5.

36. Südhof TC. (2008) Neuroligins and neurexins link synaptic function to cognitive disease. Nature 455:903-11.

37. Betancur C, Sakurai T, Buxbaum JD. (2009) The emerging role of synaptic cell-adhesion pathways in the pathogenesis of autism spectrum disorders. Trends Neurosci. 32:402-12.

38. Bourgeron T. (2009) A synaptic trek to autism. Curr Opin Neurobiol. 19:231-4.

39. Pfeiffer BE, Huber KM. (2009) The state of synapses in fragile X syndrome. Neuroscientist 15:549-67.

40. Abrahams, B.S. & Geschwind, D.H. (2008) Advances in autism genetics: on the threshold of a new neurobiology. Nat Rev Genet 9:341-355.

41. State MW. (2010) The genetics of child psychiatric disorders: focus on autism and Tourette syndrome. Neuron 68:254-69.

42. van de Lagemaat, L.N. & Grant, S.G. (2010) Genome variation and complexity in the autism spectrum. Neuron 67:8-10.

43. Kumar RA, Christian SL. (2009) Genetics of autism spectrum disorders. Curr Neurol Neurosci Rep. 9:188-97.

44. Peca J, Feliciano C, Ting JT, Wang W, Wells MF, Venkatraman TY, Lascola CD, Fu Z and Feng G. (2011) Shank3 mutant mice display autistic-like behaviours and striatal dysfunction. Nature, 472:437

45. Zhou Y, Kaiser T, Monteiro P, Zhang X, Van der Goes MS, Wang D, Barak B, Zeng M, Li C, Lu C, Wells M, Amaya A, Nguyen S, Lewis M, Sanjana N, Zhou Y, Zhang M, Zhang F, Fu Z, Feng G. (2016) Mice with Shank3 mutations associated with ASD and schizophrenia display both shared and distinct defects. Neuron 89:147-162.

46. Guo B, Chen J, Chen Q, Ren K, Feng D, Mao H, Yao H, Yang J, Liu H, Liu Y, Jia F, Qi C, Lynn-Jones T, Hu H, Fu Z, Feng G*, Wang W*, Wu S*. (2019) Anterior cingulate cortex dysfunction underlies social deficits in Shank3 mutant mice. Nat Neurosci. 22(8):1223-1234. *co-corresponding authors.

47. Zhou Y, Sharma J, Ke Q, Landman R, Yuan J, Chen H, Hayden DS, Fisher JW 3rd, Jiang M, Menegas W, Aida T, Yan T, Zou Y, Xu D, Parmar S, Hyman JB, Fanucci-Kiss A, Meisner O, Wang D, Huang Y, Li Y, Bai Y, Ji W, Lai X, Li W, Huang L, Lu Z, Wang L, Anteraper SA, Sur M, Zhou H*, Xiang AP*, Desimone R, Feng G*, Yang S*. (2019) Atypical behaviour and connectivity in SHANK3-mutant macaques. Nature. 570:326-331. *co-corresponding authors.

48. Chen Q, Deister CA, Gao X, Guo B, Lynn-Jones T, Chen N, Wells MF, Liu R, Goard MJ, Dimidschstein J, Feng S, Shi Y, Liao W, Lu Z, Fishell G, Moore CI#, Feng G#. (2020) Dysfunction of cortical GABAergic neurons leads to sensory hyper-reactivity in a Shank3 mouse model of ASD. Nat Neurosci. 23:520-532. #corresponding authors.

49. Wang W, Li C, Chen Q, van der Goes MS, Hawrot J, Yao AY, Gao X, Lu C, Zang Y, Zhang Q, Lyman K, Wang D, Guo B, Wu S, Gerfen CR, Fu Z#, Feng G# (2017) Striatopallidal dysfunction underlies repetitive behavior in Shank3-deficient model of autism. J Clin Invest. pii: 87997. #co-corresponding authors.

50. Mei Y, Monteiro P, Zhou Y, Kim J-A, Gao X, Fu Z and Feng G. (2016) Adult Restoration of Shank3 Expression Rescues Selective Autistic-Like Phenotypes. Nature, 530:481-4.

51. Segal, M., Greenberger, V. & Korkotian, E. Formation of dendritic spines in cultured striatal neurons depends on excitatory afferent activity. Eur. J. Neurosci. 17, 2573-2585 (2003)

52. Tian, X., Kai, L., Hockberger, P. E., Wokosin, D. L. & Surmeier, D. J. medium spiny neurons. 44, 94-108 (2011).

53. Zhang, Q. et al. Impaired dendritic development and memory in sorbs2 knock-out mice. J. Neurosci. 36, 2247-2260 (2016).

54. Luh, L. M., Das, I. & Bertolotti, A. qMotor, a set of rules for sensitive, robust and quantitative measurement of motor performance in mice. Nat. Protoc. 12, 1451-1457 (2017).

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

What is claimed is:

1. A non-naturally occurring polynucleotide encoding a Shank3 protein,
wherein the Shank3 protein comprises an SH3 domain, a PDZ domain, a Homer binding domain, a Cortactin binding domain, and a SAM domain;
wherein the SH3 domain comprises at least 90% identity to residues 474-525 of SEQ ID NO: 6 or at least 90% identity to residues 473-524 of SEQ ID NO: 5;
wherein the PDZ domain comprises at least 90% identity to residues 573-662 of SEQ ID NO: 6 or at least 90% identity to residues 572-661 of SEQ ID NO: 5;
wherein the Homer binding domain comprises at least 90% identity to residues 1294-1323 of SEQ ID NO: 5 or 6;
wherein the Cortactin binding domain comprises at least 90% identity to residues 1400-1426 of SEQ ID NO: 5 or 6 and/or
wherein the SAM domain comprises at least 90% identity to residues 1664-1729 of SEQ ID NO: 6 or at least 90% identity to residues 1663-1728 of SEQ ID NO:5; and
wherein the polynucleotide is less than 4.7 kb.

2. The polynucleotide of claim 1, wherein the polynucleotide further comprises a proline-rich region.

3. The polynucleotide of claim 1 or 2, wherein the SH3 domain comprises residues 474-525 of SEQ ID NO: 6 or residues 473-524 of SEQ ID NO: 5, the PDZ domain comprises residues 573-662 of SEQ ID NO: 6 or residues 572-661 of SEQ ID NO: 5, the Homer binding domain comprises residues 1294-1323 of SEQ ID NO: 5 or 6; the Cortactin binding domain comprises residues 1400-1426 of SEQ ID NO: 5 or 6 and/or the SAM domain comprises residues 1664-1729 of SEQ ID NO: 6 or residues 1663-1728 of SEQ ID NO:5.

4. The polynucleotide of any one of claims 1-3, wherein the polynucleotide comprises at least 90% identity to SEQ ID NO: 1 or 2.

5. The polynucleotide of claim 4, wherein the polynucleotide comprises SEQ ID NO: 1 or 2.

6. The polynucleotide of claim 1, wherein the Shank3 protein encoded by the polynucleotide further comprises an ankyrin repeat domain.

7. The polynucleotide of claim 6, wherein the ankyrin repeat domain comprises at least 90% identity to residues 148-345 of SEQ ID NO: 6 or at least 90% identity to residues 147-313 of SEQ ID NO: 5.

8. The polynucleotide of claim 7, wherein the ankyrin repeat domain comprises residues 148-345 of SEQ ID NO: 6 or residues 147-313 of SEQ ID NO: 5.

9. The polynucleotide of any one of claims 6-8, wherein the polynucleotide comprises at least 90% identity to SEQ ID NO: 3 or 4.

10. The polynucleotide of claim 9, wherein the polynucleotide comprises SEQ ID NO: 3 or 4.

11. The polynucleotide of any one of claims 1-10, wherein the polynucleotide is less than about 4.6 kb, 4.5 kb, 4.4 kb, 4.3 kb, 4.2 kb, 4.1 kb, 4.0 kb, 3.9 kb, 3.8 kb, 3.7 kb, 3.6 kb, 3.5 kb, 3.4 kb, 3.3 kb, 3.2 kb, 3.1 kb, 3.0 kb, 2.9 kb, 2.8 kb, 2.7 kb, 2.6 kb, 2.5 kb, 2.4 kb, 2.3 kb, 2.2 kb, or 2.1 kb.

12. The polynucleotide of any of claims 1-11, wherein the Shank3 protein is less than 65% identical to SEQ ID NO: 5 or 6 over the full length of SEQ ID NO: 5 or 6.

13. The polynucleotide of any of claims 1-12, wherein the Shank3 protein comprises an amino acid sequence that is at least 90% identical to any one of SEQ ID NOs: 17-20.

14. The polynucleotide of claim 13, wherein the Shank3 protein comprises the amino acid sequence of any one of SEQ ID NOs: 17-20.

15. A Shank3 protein encoded by a polypeptide of any one of claims 1-14.

16. A vector comprising the polynucleotide of any one of claims 1-14.

17. The vector of claim 16, wherein the vector is a viral vector.

18. The vector of claim 17, wherein the vector is an AAV vector.

19. The AAV vector of claim 18, wherein the vector comprises a promoter operably linked to the polynucleotide of any one of claims 1-14.

20. The AAV vector of claim 19, wherein the polynucleotide is flanked by AAV inverted terminal repeat (ITRs).

21. The AAV vector of any one of claims 18-20, wherein the AAV vector comprises a sequence that is at least 90% identical to SEQ ID NO: 7 or 21.

22. The AAV vector of claim 21, wherein the AAV vector comprises the sequence of SEQ ID NO: 7 or 21.

23. An AAV particle comprising an AAV vector of any one of claims 19-21 and a capsid protein, wherein the capsid is of a serotype selected from AAV1, 2, 5, 6, 8, 9, rh10, and PHP.eB.

24. The AAV particle of claim 23, wherein the serotype is AAV9.

25. The AAV particle of claim 23, wherein the serotype is AAV10.

26. The AAV particle of claim 23, wherein the serotype is AAV9-PHP.eB serotype.

27. The AAV vector of claim 19, wherein the promoter is a human promoter.

28. The AAV vector of claim 27, wherein the promoter is hSyn1.

29. A method comprising administering the AAV vector of any one of claims 18-21 or the AAV particle of claims 23-26 to a subject in need thereof.

30. The method of claim 29, wherein the AAV vector or particle is administered intravenously.

31. The method of claim 29, wherein the AAV vector or particle is delivered to the brain of the subject.

32. The method of claim 29, wherein the AAV vector or particle is delivered to the cortex, striatum and/or thalamus of the subject.

33. The method of claim 29, wherein the subject is a human subject.

34. The method of claim 33, wherein the human subject is an adult.

35. The method of claim 33, wherein the human subject is not an adult.

36. The method of claim 334, wherein the human subject is not older than 25 years old.

37. The method of claim 35, wherein the human subject is 10 years old or younger.

38. The method of any one of claims 28-36, wherein the subject has, is suspected of having, or is at risk of having, a neurodevelopmental disorder.

39. The method of any one of claims 29-37, wherein the subject has, is suspected of having, or is at risk of having, an autism spectrum disorder (ASD).

40. The method of any one of claims 29-37, wherein the subject exhibits one or more symptoms of an ASD.

41. The method of any one of claims 29-37, wherein the subject has, is suspected of having, or is at risk of having, Phelan-McDermid syndrome.

42. The method of any one of claims 29-41, wherein the subject exhibits one or more of: developmental delay, intellectual disability (ID), sleep disturbance, hypotonia, lack of speech, or language delay.

43. The method of any one of claims 29-42, wherein the subject has, is suspected of having, or is at risk of having, reduced expression of the Shank3 gene relative to a control subject.

44. The method of claim 43, wherein the control subject is a subject that does not have, is not suspected of having, or is not at risk of having, a neurodevelopmental disorder, an autism spectrum disorder (ASD), and/or Phelan-McDermid syndrome.

45. The method of claim 43 or 44, wherein reduced expression of the Shank3 gene is caused by disruption of at least one copy of the Shank3 gene.

46. The method of claim 45, wherein disruption of the Shank3 gene comprises a deletion in at least one copy of the Shank3 gene.

47. The method of claim 45, wherein disruption of the Shank3 gene comprises one or more mutations within at least one copy of the Shank3 gene.

48. A method of treating a subject having a neurodevelopmental disorder, the method comprising administering to the subject an effective amount of a composition comprising an AAV vector, wherein the AAV vector comprises a polynucleotide encoding a Shank3 protein.

49. A method of treating a subject having an autism spectrum disorder (ASD), the method comprising administering to the subject an effective amount of a composition comprising an AAV vector, wherein the AAV vector comprises a polynucleotide encoding a Shank3 protein.

50. A method of treating a subject having Phelan-McDermid syndrome, the method comprising administering to the subject an effective amount of a composition comprising an AAV vector, wherein the AAV vector comprises a polynucleotide encoding a Shank3 protein.

51. The method of any one of claims 48-50, wherein the Shank3 protein comprises an SH3 domain, a PDZ domain, a Homer binding domain, a Cortactin binding domain, and a SAM domain.

52. The method of any one of claims 48-50, wherein the Shank3 protein further comprises an ankyrin repeat domain.

53. The method of claim 52, wherein the ankyrin repeat domain comprises at least 90% identity to residues 148-345 of SEQ ID NO: 6 or at least 90% identity to residues 147-313 of SEQ ID NO: 5.

54. The method of any one of claims 51-53, wherein the SH3 domain comprises at least 90% identity to residues 474-525 of SEQ ID NO: 6 or at least 90% identity to residues 473-524 of SEQ ID NO: 5;
wherein the PDZ domain comprises at least 90% identity to residues 573-662 of SEQ ID NO: 6 or at least 90% identity to residues 572-661 of SEQ ID NO: 5;
wherein the Homer binding domain comprises at least 90% identity to residues 1294 1323 of SEQ ID NO: 5 or 6;
wherein the Cortactin binding domain comprises at least 90% identity to residues 1400-1426 of SEQ ID NO: 5 or 6; and/or
wherein the SAM domain comprises at least 90% identity to residues 1664-1729 of SEQ ID NO: 6 or at least 90% identity to residues 1663-1728 of SEQ ID NO:5.

55. The method of any one of claims 48-50, wherein the SH3 domain comprises residues 474-525 of SEQ ID NO: 6 or residues 473-524 of SEQ ID NO: 5,
wherein the PDZ domain comprises residues 573-662 of SEQ ID NO: 6 or residues 572-661 of SEQ ID NO: 5,
wherein the Homer binding domain comprises residues 1294-1323 of SEQ ID NO: 5 or 6;
wherein the Cortactin binding domain comprises residues 1400-1426 of SEQ ID NO: 5 or 6; and/or
wherein the SAM domain comprises residues 1664-1729 of SEQ ID NO: 6 or residues 1663-1728 of SEQ ID NO:5.

56. The method of any one of claims 47-49, wherein the polynucleotide is less than 4.7 kb.

57. The method of any one of claims 48-50, wherein the polynucleotide is less than about 4.6 kb, 4.5 kb, 4.4 kb, 4.3 kb, 4.2 kb, 4.1 kb, 4.0 kb, 3.9 kb, 3.8 kb, 3.7 kb, 3.6 kb, 3.5 kb, 3.4 kb, 3.3 kb, 3.2 kb, 3.1 kb, 3.0 kb, 2.9 kb, 2.8 kb, 2.7 kb, 2.6 kb, 2.5 kb, 2.4 kb, 2.3 kb, 2.2 kb, or 2.1 kb.

58. The method of any one of claims 48-50, wherein the polynucleotide further comprises a proline-rich region.

59. The method of any one of claims 48-50, wherein the polynucleotide comprises at least 90% identity to any one of SEQ ID NOs: 1-4.

60. The method of any one of claims 48-50, wherein the polynucleotide comprises any one of SEQ ID NOs: 1-4.

61. The method of any one of claims 478-50, wherein the subject is a human subject.

62. The method of claim 61, wherein the human subject is an adult.

63. The method of claim 61, wherein the human subject is not an adult.

64. The method of claim 62, wherein the human subject is not older than 25 years old.

65. The method of claim 63, wherein the human subject is 10 years old or younger.

66. The method of any one of claims 48-65, wherein the composition is administered intravenously.

67. The method of any one of claims 48-65, wherein the composition is delivered to the brain of the subject.

68. The method of any one of claims 48-65, wherein the composition is delivered to the striatum and/or thalamus of the subject.

69. The method of any one of claims 48-68, wherein the subject exhibits one or more of: developmental delay, intellectual disability (ID), sleep disturbance, hypotonia, lack of speech, or language delay.

70. The method of claim 48, wherein the autism spectrum disorder (ASD) comprises autism disorder.

71. The method of any one of claims 48-70, wherein the subject has, is suspected of having, or is at risk of having, reduced expression of the Shank3 gene relative to a control subject.

72. The method of claim 71, wherein the control subject is a subject that does not have, is not suspected of having, or is not at risk of having, a neurodevelopmental disorder, an autism spectrum disorder (ASD), and/or Phelan-McDermid syndrome.

73. The method of claim 71 or 72, wherein reduced expression of the Shank3 gene is caused by disruption of at least one copy of the Shank3 gene.

74. The method of claim 73, wherein disruption of the Shank3 gene comprises a deletion in at least one copy of the Shank3 gene.

75. The method of claim 73, wherein disruption of the Shank3 gene comprises one or more mutations within at least one copy of the Shank3 gene.

76. The method of any one of claims 48-50, wherein the subject has improved sleep efficiency after being administered to an effective amount of the composition.

77. The method of any one of claims 48-76, wherein the composition is in a pharmaceutically acceptable carrier.

78. A MiniShank3 protein comprising a sequence that is at least 80%, at least 85%, at least 90%, or at least 95% identical to any one of SEQ ID NOs: 17-20.

79. The MiniShank3 protein of claim 78 wherein the MiniShank3 protein comprises the sequence of any one of SEQ ID NOs: 17-20.


SEQUENCE LISTING (22 sequences)

SEQ ID NO: 1 - DNA, 2121 bp, Artificial Sequence
  Synthetic
atgggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga

SEQ ID NO: 2 - DNA, 2124 bp, Artificial Sequence
  Synthetic
atgggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga

SEQ ID NO: 3 - DNA, 3276 bp, Artificial Sequence
  Synthetic
atggacggccccggggccagcgccgtggtcgtgcgcgtcggcatcccggacctgcaacaaacgaagtgcctgcgtctggacccaaccgcgcccgtgtgggccgccaagcagcgtgtgctctgcgccctcaatcatagccttcaagacgcgctcaactacgggctattccagcctccctcccggggtcgcgccggcaagttcctggatgaagagcggctcttacaggactacccgcctaacctggacacgcccctgccctatctggagttccgatacaagcggagagtttatgcccagaacctcatagatgacaagcagtttgcaaagctacacacaaaggcaaacctgaagaagttcatggactatgtccagctacacagcacagataaggtggcccgcctgctggacaaggggctggaccccaatttccatgaccctgactcaggagagtgccctctgagccttgcggcacagttggacaacgccactgacctcctgaaggttctccgcaacggcggtgctcatctggacttccggacccgagatgggctgacagccgtccactgtgctacccgccagcggaacgcaggggcattgacgaccctgctggacctgggggcttcgcctgactacaaggacagccgcggcctgacgcccctgtaccatagtgccctagggggcggggatgccctctgttgcgagctgcttctccatgatcatgcacagctggggaccactgatgagaatggttggcaagagatccatcaggcctgtcgctttggacacgtgcagcacctggagcaccttttgttctatggggccaacatgggtgctcagaatgcctcgggaaacacagccctgcacatctgtgccctctacaaccaggagagttgcgcgcgcgtcctgcttttccgtggtgccaacaaggacgtccgcaattacaacagccagacagccttccaggtggccattattgcagggaactttgagcttgccgaggtaatcaagacccacaaagactccgatgtcgtaccattcagggaaacccccagctatgcaaagcgacggcgtctggctggcccgagtggcctggcatccccacggcccttacagcgctcagccagtgatatcaacctgaaaggtgcgccgccgccccggggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga

SEQ ID NO: 4 - DNA, 3279 bp, Artificial Sequence
  Synthetic
atggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatgcgaagcggcggcgactggctggccccagtggcttggcatcccctcggcctctgcagcgctcagccagcgatatcaacctgaagggggcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga

SEQ ID NO: 5 - protein, 1730 aa, Mus musculus
MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGDQPAASPGPTLRSLPHQLLLQRLQEEKDRDRDGELENDISGPSAGRGGHNKISPSGPGGSGPAPGPGPASPAPPAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRRRKGEKLDEILAVAAEPTLRPDIADADSRAATVKQRPTSRRITPAEISSLFERQGLPGPEKLPGSLRKGIPRTKSVGEDEKLASLLEGRFPRSTSMQDTVREGRGIPPPPQTAPPPPPAPYYFDSGPPPTFSPPPPPGRAYDTVRSSFKPGLEARLGAGAAGLYDPSTPLGPLPYPERQKRARSMIILQDSAPEVGDVPRPAPAATPPERPKRRPRPSGPDSPYANLGAFSASLFAPSKPQRRKSPLVKQLQVEDAQERAALAVGSPGPVGGSFAREPSPTHRGPRPGSLDYSSGEGLGLTFGGPSPGPVKERRLEERRRSTVFLSVGAIEGSPPSADLPSLQPSRSIDERLLGTGATTGRDLLLPSPVSALKPLVGGPSLGPSGSTFIHPLTGKPLDPSSPLALALAARERALASQTPSRSPTPVHSPDADRPGPLFVDVQTRDSERGPLASPAFSPRSPAWIPVPARREAEKPPREERKSPEDKKSMILSVLDTSLQRPAGLIVVHATSNGQEPSRLGAEEERPGTPELAPAPMQAAAVAEPMPSPRAQPPGSIPADPGPGQGSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 6 - protein, 1731 aa, Homo sapiens
MDGPGASAVVVRVGIPDLQQTKCLRLDPAAPVWAAKQRVLCALNHSLQDALNYGLFQPPSRGRAGKFLDEERLLQEYPPNLDTPLPYLEFRYKRRVYAQNLIDDKQFAKLHTKANLKKFMDYVQLHSTDKVARLLDKGLDPNFHDPDSGECPLSLAAQLDNATDLLKVLKNGGAHLDFRTRDGLTAVHCATRQRNAAALTTLLDLGASPDYKDSRGLTPLYHSALGGGDALCCELLLHDHAQLGITDENGWQEIHQACRFGHVQHLEHLLFYGADMGAQNASGNTALHICALYNQESCARVLLFRGANRDVRNYNSQTAFQVAIIAGNFELAEVIKTHKDSDVVPFRETPSYAKRRRLAGPSGLASPRPLQRSASDINLKGEAQPAASPGPSLRSLPHQLLLQRLQEEKDRDRDADQESNISGPLAGRAGQSKISPSGPGGPGPAPGPGPAPPAPPAPPPRGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRRRKGEKLDEMLAAAAEPTLRPDIADADSRAATVKQRPTSRRITPAEISSLFERQGLPGPEKLPGSLRKGIPRTKSVGEDEKLASLLEGRFPRSTSMQDPVREGRGIPPPPQTAPPPPPAPYYFDSGPPPAFSPPPPPGRAYDTVRSSFKPGLEARLGAGAAGLYEPGAALGPLPYPERQKRARSMIILQDSAPESGDAPRPPPAATPPERPKRRPRPPGPDSPYANLGAFSASLFAPSKPQRRKSPLVKQLQVEDAQERAALAVGSPGPGGGSFAREPSPTHRGPRPGGLDYGAGDGPGLAFGGPGPAKDRRLEERRRSTVFLSVGAIEGSAPGADLPSLQPSRSIDERLLGTGPTAGRDLLLPSPVSALKPLVSGPSLGPSGSTFIHPLTGKPLDPSSPLALALAARERALASQAPSRSPTPVHSPDADRPGPLFVDVQARDPERGSLASPAFSPRSPAWIPVPARREAEKVPREERKSPEDKKSMILSVLDTSLQRPAGLIVVHATSNGQEPSRLGGAEEERPGTPELAPAPMQSAAVAEPLPSPRAQPPGGTPADAGPGQGSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 7 - DNA, 7459 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcagcgctgttaacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggaggaattcacggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctgagcggccatcgtcgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 5331 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcagcgctgttaacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 6369 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcaccatgcctaagaagaagagaaaggtatgcaggtcacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatgttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 5703 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcaccatgcctaagaagaagagaaaggtatgcaggtcacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgagttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - protein, 950 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGGIHGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 12 - protein, 239 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13 - protein, 587 aa, Artificial Sequence
  Synthetic
MPKKKRKVCRSHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDVNVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 14 - protein, 365 aa, Artificial Sequence
  Synthetic
MPKKKRKVCRSHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRVNVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - DNA, 5193 bp, Mus musculus
atggacggccccggggccagcgccgtggtcgtgcgcgtcggcatcccggacctgcaacaaacgaagtgcctgcgtctggacccaaccgcgcccgtgtgggccgccaagcagcgtgtgctctgcgccctcaatcatagccttcaagacgcgctcaactacgggctattccagcctccctcccggggtcgcgccggcaagttcctggatgaagagcggctcttacaggactacccgcctaacctggacacgcccctgccctatctggagttccgatacaagcggagagtttatgcccagaacctcatagatgacaagcagtttgcaaagctacacacaaaggcaaacctgaagaagttcatggactatgtccagctacacagcacagataaggtggcccgcctgctggacaaggggctggaccccaatttccatgaccctgactcaggagagtgccctctgagccttgcggcacagttggacaacgccactgacctcctgaaggttctccgcaacggcggtgctcatctggacttccggacccgagatgggctgacagccgtccactgtgctacccgccagcggaacgcaggggcattgacgaccctgctggacctgggggcttcgcctgactacaaggacagccgcggcctgacgcccctgtaccatagtgccctagggggcggggatgccctctgttgcgagctgcttctccatgatcatgcacagctggggaccactgatgagaatggttggcaagagatccatcaggcctgtcgctttggacacgtgcagcacctggagcaccttttgttctatggggccaacatgggtgctcagaatgcctcgggaaacacagccctgcacatctgtgccctctacaaccaggagagttgcgcgcgcgtcctgcttttccgtggtgccaacaaggacgtccgcaattacaacagccagacagccttccaggtggccattattgcagggaactttgagcttgccgaggtaatcaagacccacaaagactccgatgtcgtaccattcagggaaacccccagctatgcaaagcgacggcgtctggctggcccgagtggcctggcatccccacggcccttacagcgctcagccagtgatatcaacctgaaaggtgatcagcccgcagcttctccagggcccactctccgaagcctccctcatcaactcttgctccagaggcttcaggaggagaaagaccgtgacagggatggtgaactggagaatgacatcagcggcccctcagcaggcaggggtggccacaacaagatcagccccagtgggcccggcggatccggccccgcgcccggccccggcccggcgtctcccgcgccccccgcgccgccgccccggggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagaagaaaaggggagaagttggatgagatcctggcagttgccgcggagccgacactgaggccggacattgcagatgctgactcgagggcggccactgtcaagcagcggcccaccagccggaggatcacccctgctgagatcagctcattgtttgagcgccagggcctcccaggcccagagaagctgccgggctctctgcggaaggggattccacggaccaaatctgtaggggaggatgagaagctggcatccctactggaagggcgcttcccacgcagcacgtcaatgcaggacacagtgcgtgaaggtcgaggcattccacccccgccgcagaccgccccgccacccccacccgcgccctactacttcgactccgggccaccccccaccttctcaccgccgccaccaccgggccgggcctatgacactgtgcgctccagcttcaaaccaggcctggaggctcgtctgggtgcgggggccgccggcctgtatgatccgagcacgcctctgggcccgctgccctaccctgagcgtcagaagcgtgcgcgctccatgatcatactgcaggactctgcgccagaagtgggtgatgtcccccggcctgcgcctgccgccacaccgcctgagcgccccaagcgccggcctcggccgtcaggccctgatagtccctatgccaacctgggcgccttcagtgccagcctcttcgctccgtcgaaaccacagcgccgcaagagcccgctggtgaagcagcttcaggtggaggacgctcaggagcgcgcggccctggccgtgggtagcccgggaccagtgggcggaagctttgcacgagaaccctctccgacgcaccgcgggccccggccaggcagccttgactacagctctggagaaggcctaggccttaccttcggcggccctagccctggcccagtcaaggagcggcgcctggaggagcgacgccgttccactgtgttcctctctgtgggtgccatcgagggcagccctcccagcgcggatctgccatccctacaaccctcccgctccattgatgagcgcctcctggggacaggcgccaccactggccgcgatttgctactcccctcccctgtctctgctctgaagccattggtcggtggtcccagccttgggccctcaggctccaccttcatccaccctctcactggcaaacccttggatcctagctcacccttagctcttgctctggctgcccgggagcgggctctggcctcgcaaacaccttcccggtcccccacacctgtgcacagccccgatgctgaccgccctggacccctctttgtggatgtgcaaacccgagactctgagagaggaccgttggcttccccagccttctcccctcggagtccagcgtggattccagtgcctgctcggagagaggcagagaagccccctcgggaagagcggaagtcaccagaggacaagaagtccatgatcctcagcgtcttggacacgtccttgcaacggccagctggcctcattgttgtgcatgccaccagcaatgggcaggagcccagcaggctgggggctgaagaggagcgccccggtactccggagctggccccagcccccatgcaggcagcagctgtggcagagcccatgccaagcccccgggcccagccccctggcagcatcccagcagatcccgggccaggtcaaggcagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga

SEQ ID NO: 16 - DNA, 5421 bp, Homo sapiens
atgcagctgagccgcgccgccgccgccgccgccgccgcccctgcggagcccccggagccgctgtcccccgcgccggccccggccccggccccccccggccccctcccgcgcagcgcggccgacggggctccggcgggggggaagggggggccggggcgccgcgcggagtccccgggcgctccgttccccggcgcgagcggccccggcccgggccccggcgcggggatggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatgcgaagcggcggcgactggctggccccagtggcttggcatcccctcggcctctgcagcgctcagccagcgatatcaacctgaagggggaggcacagccagcagcttctcctggaccctcgctgagaagcctcccccaccagctgctgctccagcggctgcaagaggagaaagatcgtgaccgggatgccgaccaggagagcaacatcagtggccctttagcaggcagggccggccaaagcaagatcagcccgagcgggcccggcggccccggccccgcgcccggccccggccccgcgccccctgcgccccccgcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagaagaaaaggggagaagctggacgagatgctggcagccgccgcagagccaacgctgcggccagacatcgcagacgcagactccagagccgccaccgtcaaacagaggcccaccagtcggaggatcacacccgccgagattagctcattgtttgaacgccagggcctcccaggcccagagaagctgccgggctccttgcggaaggggattccacggaccaagtctgtaggggaggacgagaagctggcgtccctgctggaagggcgcttcccgcggagcacctcgatgcaagacccggtgcgcgagggtcgcggcatcccgcccccgccgcagaccgcgccgcctcccccgcccgcgccctactacttcgactcggggccgcccccggccttctcgccgccgcccccgccgggccgcgcctacgacacggtgcgctccagcttcaagcccggcctggaggcgcgcctgggcgcgggcgctgccggcctgtacgagccgggcgcggccctcggcccgctgccgtatcccgagcggcagaagcgcgcgcgctccatgatcatcctgcaggactcggcgcccgagtcgggcgacgcccctcgacccccgcccgcggccaccccgcccgagcgacccaagcgccggccgcggccgcccggccccgacagcccctacgccaacctgggcgccttcagcgccagcctcttcgctccgtccaagccgcagcgccgcaagagccccctggtgaagcagctgcaggtggaggacgcgcaggagcgcgcggccctggccgtgggcagccccggtcccggcggcggcagcttcgcccgcgagccctccccgacccaccgcggtccgcgcccgggtggcctcgactacggcgcgggcgatggcccggggctcgcgttcggcggcccgggcccggccaaggaccggcggctggaggagcggcgccgctccactgtgttcctgtccgtgggggccatcgagggcagcgcccccggcgcggatctgccatccctacagccctcccgctccatcgacgagcgcctcctggggaccggccccaccgccggccgcgacctgctgctgccctccccggtgtctgccctgaagccgttggtcagcggcccgagcctggggccctcgggttccaccttcatccacccactcaccggcaaacccctggaccccagctcacccctggcccttgccctggctgcccgagagcgagctctggcctcccaggcgccctcccggtcccccacacccgtgcacagtcccgacgccgaccgccccggacccctgtttgtggatgtacaggcccgggacccagagcgagggtccctggcttccccggctttctccccacggagcccagcctggattcctgtgcctgctcgcagggaggcagagaaggtcccccgggaggagcggaagtcacccgaggacaagaagtccatgatcctcagcgtcctggacacatccctgcagcggccagctggcctcatcgttgtgcacgccaccagcaacgggcaggagcccagcaggctggggggggccgaagaggagcgcccgggcaccccggagttggccccggcccccatgcagtcagcggctgtggcagagcccctgcccagcccccgggcccagccccctggtggcaccccggcagacgccgggccaggccagggcagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga

SEQ ID NO: 17 - protein, 706 aa, Artificial Sequence
  Synthetic
MGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 18 - protein, 707 aa, Artificial Sequence
  Synthetic
MGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 19 - protein, 1091 aa, Artificial Sequence
  Synthetic
MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 20 - protein, 1092 aa, Artificial Sequence
  Synthetic
MDGPGASAVVVRVGIPDLQQTKCLRLDPAAPVWAAKQRVLCALNHSLQDALNYGLFQPPSRGRAGKFLDEERLLQEYPPNLDTPLPYLEFRYKRRVYAQNLIDDKQFAKLHTKANLKKFMDYVQLHSTDKVARLLDKGLDPNFHDPDSGECPLSLAAQLDNATDLLKVLKNGGAHLDFRTRDGLTAVHCATRQRNAAALTTLLDLGASPDYKDSRGLTPLYHSALGGGDALCCELLLHDHAQLGITDENGWQEIHQACRFGHVQHLEHLLFYGADMGAQNASGNTALHICALYNQESCARVLLFRGANRDVRNYNSQTAFQVAIIAGNFELAEVIKTHKDSDVVPFRETPSYAKRRRLAGPSGLASPRPLQRSASDINLKGAPPPRGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 21 - DNA, 6701 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcatgggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 495 bp, Homo sapiens
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctaga